(19) United States
(12) Patent Application Publication
(10) Pub. No.: US 2024/0153590 A1
(43) Pub. Date: May 9, 2024

(54) NATURAL LANGUAGE PROCESSING TO PREDICT PROPERTIES OF PROTEINS

(71) Applicant: GLAXOSMITHKLINE BIOLOGICALS SA, Rixensart (BE)

(72) Inventors: Ahmed ESSAGHIR, Rixensart (BE); Gurpreet SINGH, Collegeville, PA (US); Paul SMYTH, Rixensart (BE)

(73) Assignee: GLAXOSMITHKLINE BIOLOGICALS SA, Rixensart (BE)

(21) Appl. No.: 18/279,526

(22) PCT Filed: Feb. 28, 2022
(86) PCT No.: PCT/IB2022/051740

§ 371 Date: Aug. 30, 2023

Related U.S. Application Data
(60) Provisional application No. 63/155,506, filed on Mar. 2, 2021.

(51) Int. Cl. G16B 40/20; G06F 40/284; G06F 40/40; G16B 15/20

(52) U.S. Cl. CPC G16B 40/20; G06F 40/284; G06F 40/40; G16B 15/20

(57) ABSTRACT

A protein language natural language processing (NLP) system is trained to predict specific biophysiochemical properties. Amino acids of proteins are tokenized and masked. A first neural network is trained on a library of amino acid sequences in an unsupervised or self-supervised manner. The information obtained from the first phase of training is applied in a subsequent training operation via transfer learning, to a second neural network. In aspects, an annotated compact dataset is used to fine-tune the second neural network in a second phase of training, and in a supervised manner, to predict biophysiochemical properties of proteins, including TCR-epitope binding.

### FIELD OF THE INVENTION

The present application relates to predicting properties of a protein using natural language processing, and more specifically, to methods, systems and computer-readable media for utilizing natural language processing to predict physical, biological, and/or chemical properties of a protein.

### BACKGROUND

Various in silico and in vitro approaches have been developed to analyze the structural and functional features of proteins. In vitro approaches aim to understand protein structure and function using experimental techniques. For example, proteins may be synthesized, crystallized, and analyzed based on their crystal structure or characterized with various binding assays, expression assays, motility assays, luminescence assays or mechanical assays. However, such wet-lab based approaches are costly and time consuming.

De novo in silico approaches attempt to predict the second, third, and even fourth dimensional structures and corresponding functions of a protein from its primary amino acid structure by simulation, for example, using molecular dynamics simulations. However, such approaches typically have a high computational cost, are time consuming, and may not generate structures that correspond well with known biological structures. While improvements in computing architecture such as distributed computing/massively parallel super computing have increased computational power, these approaches are still costly and time-consuming to build and may be shared among multiple research and scientific groups which may limit access.

More recently, machine learning driven in-silico approaches have emerged, offering alternatives to traditional time-consuming de novo computational approaches while being far less cost prohibitive than wet-lab approaches. However, there are disadvantages to machine learning techniques. Machine learning approaches may be tailored to one application with limited or no transferability to other applications, may require a large amount of expert-annotated data for a particular task, and may rely on time-consuming, trial-and-error processes of feature design and parameter selection. Thus, early generation machine learning approaches replaced time consuming and costly wet-lab work with time-consuming, expensive, trial-and-error based computational techniques, trained to analyze a single biological topic.

Recent advances in natural language processing (NLP) have led to alternatives to generating a large expert-annotated dataset. By applying self-supervised learning to train an NLP model on a repository of English text documents that adheres to a grammatical standard, the NLP system learns the lexicography of a particular language without a large, expert-annotated training dataset. In aspects, the trained NLP model may be subsequently retrained for another language task such as question answering (see, Devlin et al., BERT: Pre-training of Deep Bidirectional Transformers for Language Understanding (2019), arxiv.org/pdf/1810.04805.pdf). In other aspects, NLP approaches have been utilized for binding predictions (see, Filipavicius et al., “Pre-training Protein Language Models with Label-Agnostic Binding Pairs Enhances Performance in Downstream Tasks” (2020), https://arxiv.org/abs/2012.03084). While such approaches have worked well for natural languages having defined grammatical rules relating to sentence structure and parts of speech, the applicability of such approaches in other areas is not well understood.

There is an ongoing need for computational approaches having the capability to accurately predict various biological, physical, and chemical properties that can be performed within a suitable timeframe with accessible computing resources and with increased accuracy.

### SUMMARY

The following paragraphs provide a summary of various aspects of training and using an NLP system to predict biophysiochemical properties of an amino acid sequence using natural language processing (NLP). The summary is not to be limited to the following exemplary embodiments.

### Training the NLP System

In an embodiment, a computer-implemented method for training a predictive protein language NLP system to predict biophysiochemical properties of an amino acid sequence using natural language processing (NLP) is provided comprising:
in a first phase, training the predictive protein language NLP system with a diversified protein sequence dataset in a self-supervised manner; and
in a second phase, training the predictive protein language NLP system, wherein the protein language NLP system comprises features from the first phase of training In aspects, training in a second phase may include training with an annotated protein sequence dataset in a supervised manner, wherein the annotated protein sequence dataset comprises individual protein sequences that are annotated with one or more biophysiochemical properties.

In an aspect, the method further comprises training, in the first phase, the predictive protein language NLP system using a diversified protein sequence dataset that has undergone individual amino acid-level tokenization, n-mer level tokenization, or sub-word level tokenization of respective protein sequences.

In another aspect, the method further comprises: training, in the first phase, the predictive protein language NLP system using a diversified protein sequence dataset, wherein about 5-25%, 10-20%, 12-17% or 15% of the amino acids in the diversified protein sequence dataset are masked.

In an aspect, the method further comprises: training the predictive protein language NLP system, wherein the NLP system comprises a first neural network that is trained in the first phase and a second neural network comprising features from the first neural network that is trained in the second phase.

In another aspect, the first neural network comprises at least one transformer model (e.g., a first transformer model and a second transformer model) having at least one encoder and at least one decoder.

In still another aspect, the first transformer model comprises a first transformer model with attention (e.g., self-attention). In still another aspect, the second transformer model comprises a second transformer model with attention (e.g., self-attention).

In another aspect, the second neural network comprises a neural network (e.g., a perceptron, a transformer model having at least one encoder and at least one decoder, etc.).

In an aspect, the transformer model with attention further comprises a robustly optimized bidirectional encoder representations from transformers model (e.g., a RoBERTa model).

In another aspect, the first and/or second neural network comprises a long short term memory (LSTM) model.

In still another aspect, the method further comprises:
training the first neural network with the diversified protein sequence dataset until meeting a first criterion; and
storing features associated with the trained first neural network in memory. In aspects, stored features may include but are not limited to the configuration and parameters of the first neural network, including a model type, a number of layers, weights, inputs, outputs, hyperparameters, optimizer, etc. In aspects, the stored features allow a user to reconstruct the trained first neural network or a portion thereof, or to transfer knowledge from the training of the first neural network to the second neural network.

In still another aspect, the method further comprises:
obtaining a second neural network comprising features of the trained first neural network;
modifying the second neural network (e.g., by truncating one or more layers of the second neural network, and replacing the truncated layers with one or more replacement layers); and training the modified second neural network (e.g., with an annotated protein sequence dataset to predict one or more biophysiochemical properties), until meeting a second criterion.

In still another aspect, the method further comprises: transferring the information associated with the trained first neural network to a second neural network. In some aspects, and in reference to transfer learning, the second neural network may be modified by truncating one or more output layers of the neural network, and replacing the truncated layers with one or more replacement layers (untrained layers). In some aspects, the modified neural network is trained, until meeting a second criterion, with the annotated protein sequence dataset to predict one or more specific biophysiochemical properties. In other aspects, the second neural network may be provided with embeddings (e.g., embeddings, concatenated representations of sequence and categorical feature embeddings) from the trained first neural network to facilitate information transfer from the first phase of training to the second phase of training.

In still another aspect, the second neural network may be trained to predict one or more biophysiochemical properties including but not limited to: protein expression, binding affinity of a protein to a target, stability of a protein, fluorescence (log fluorescence intensity) of a protein, crystallization of a protein, therapeutic efficacy of a protein, or biophysiochemical properties related to structure-function of the protein (e.g., cellular mechanisms, disease-causing functional changes, disease prevention, diagnosis, and treatment).

In an aspect, the method further comprises: generating, for display on a display screen, information from a salience module that indicates a contribution of respective amino acids to the prediction of the biophysiochemical property.

In an aspect, the predictive protein language NLP system may be further trained with experimental data to validate predicted biophysiochemical properties of a candidate amino acid sequence.

A computer-implemented method for predicting biophysiochemical properties of an amino acid sequence using natural language processing (NLP) comprising:
obtaining a protein language NLP system trained in a first phase on a diversified protein sequence dataset in a self-supervised manner; and
training the obtained protein language NLP system, optionally with an annotated protein sequence dataset in a supervised manner, to predict a biophysiochemical property; and
using the trained predictive protein language NLP system to predict the biophysiochemical property. In aspects, the biophysiochemical property is binding or binding affinity of TCR epitopes.

### Trained NLP System

According to an embodiment of the present techniques, a computer-implemented method for predicting biophysiochemical properties of an amino acid sequence using natural language processing (NLP) is provided comprising:

providing a trained predictive protein language NLP system:
trained, in a first phase, with a diversified protein sequence dataset in a self-supervised manner, and
trained, in a second phase and including features from the first phase (e.g., with an annotated protein sequence dataset in a supervised manner, wherein the annotated protein sequence dataset comprises individual protein sequences that are annotated with one or more biophysiochemical properties);

receiving an input query, from a user interface device coupled to the trained predictive protein language NLP system, comprising a candidate amino acid sequence; and

generating, by the trained predictive protein language NLP system, an output comprising a prediction including one or more biophysiochemical properties for the candidate amino acid sequence. In aspects, the second phase of training may comprise training with an annotated protein sequence dataset in a supervised manner, wherein the annotated protein sequence dataset comprises individual protein sequences that are annotated with one or more biophysiochemical properties. In aspects, the output is displayed (optionally), on a display screen of a device (e.g., server device, client device), the output comprising the predicted one or more biophysiochemical properties for the candidate amino acid sequence. Alternatively, information may be transferred from a first neural network to a second neural network by providing embeddings and/or sequence representations (e.g., concatenated representations of sequence and categorical feature embeddings) as input into the second neural network.

According to an embodiment, a computer-implemented method for predicting biophysiochemical properties of an amino acid sequence using natural language processing (NLP) is provided comprising:
providing a predictive protein language NLP system trained to predict a biophysiochemical property of a candidate amino acid sequence;
receiving an input query, from a user interface device coupled to the predictive protein language NLP system, comprising a candidate amino acid sequence;
generating, by the predictive protein language NLP system, an output comprising a prediction including one or more biophysiochemical properties for the candidate amino acid sequence. In aspects, the output is displayed (optionally), on a display screen of a device, the output comprising the predicted one or more biophysiochemical properties for the candidate amino acid sequence.

In an aspect, the computer-implemented method further comprises: accessing a trained predictive protein language NLP system, the NLP system trained in the first phase by masking at least a portion of individual amino acids in the diversified protein sequence dataset. In aspects, about 5-25%, 10-20%, 12-17%, or 15% of individual amino acids in the diversified protein sequence are masked.

In another aspect, the computer-implemented method further comprises: accessing a trained predictive protein language NLP system, the NLP system trained in the first phase using a diversified protein sequence dataset that has undergone individual amino acid-level tokenization, n-mer level tokenization, and sub-word level tokenization of respective protein sequences.

In another aspect, the computer-implemented method further comprises: accessing a trained predictive protein language NLP system, the NLP system trained in the first phase using a diversified protein sequence dataset, wherein about 5-25%, 10-20%, 12-17%, or 15% of the amino acids in the diversified protein sequence dataset are masked.

In an aspect, the computer-implemented method further comprises: accessing a trained predictive protein language NLP system, the NLP system generated by training in a first phase a first neural network, and in a second phase a second neural network.

In another aspect, the first neural network comprises at least one or more transformer models (e.g., a first transformer model, a second transformer model, etc.) having at least one encoder and at least one decoder.

In still another aspect, the first transformer model comprises a transformer model with attention (e.g., self-attention).

In still another aspect, the second transformer model comprises a transformer model with attention (e.g., self-attention).

In another aspect, the first and/or second transformer model with self-attention further comprises a robustly optimized bidirectional encoder representations from transformers model.

In yet another aspect, the second neural network comprises for example, a perceptron, or a transformer model having at least one encoder and at least one decoder.

In still another aspect, the first and/or second neural network comprises a LSTM model.

In another aspect, the computer-implemented method further comprises: receiving a plurality of candidate amino acid sequences generated in silico, generating a prediction of whether the respective candidate amino acid sequences have one or more biophysiochemical properties. In yet another aspect, the candidate amino acid sequences are displayed according to a ranking of the predicted one or more biophysiochemical properties.

In aspects, the output of the trained protein language NLP system may be utilized to select hypothetical/in-silico therapeutic candidates for synthesis, for example, for experimental validation of biophysiochemical predictions. In other aspects, the output of the trained protein language NLP system may be utilized to select a lead therapeutic candidate compatible with manufacturing processes (e.g., solubility, expression levels, etc.).

In still another aspect, the one or more predicted biophysiochemical properties include but are not limited to: protein expression, binding affinity of a protein to a target, stability of a protein, fluorescence (log fluorescence intensity) of a protein, crystallization of a protein, therapeutic efficacy of a protein, biophysiochemical properties related to structure-function of the protein (e.g., cellular mechanisms, disease-causing functional changes, disease prevention, diagnosis, and treatment).

The computer-implemented method further comprises: receiving a plurality of candidate amino acid sequences; analyzing the candidate amino acid sequences; and predicting whether the candidate amino acid sequences have one or more biophysical properties.

In yet another aspect, the computer-implemented method further comprises: providing for display on a display screen information from a salience module that indicates a contribution of respective amino acids to the prediction of a biophysiochemical property.

In still another aspect, the computer-implemented method further comprises: providing on the display screen information from a salience module that indicates a level of attention for each amino acid of the candidate sequence.

### Trained Executable NLP System

According to an embodiment of the present techniques, a computer-implemented method for predicting biophysiochemical properties of an amino acid sequence using natural language processing (NLP) is provided comprising:
receiving an executable program corresponding to a trained predictive protein language NLP system:
   trained, in a first phase, with a diversified protein sequence dataset in a self-supervised manner, and
   trained, in a second phase and including features from the first phase;
loading the executable program into memory and executing with one or more processors the executable program corresponding to the trained predictive protein language NLP system. Optionally, in the second phase, training may comprise using an annotated protein sequence dataset in a supervised manner, wherein the annotated protein sequence dataset comprises individual protein sequences that are annotated with one or more biophysiochemical properties.

According to an embodiment of the present techniques, a computer-implemented method or system for predicting biophysiochemical properties of an amino acid sequence using natural language processing (NLP) is provided comprising:
receiving an executable program corresponding to a trained predictive protein language NLP system; and
loading the executable program into memory and executing with one or more processors the executable program corresponding to the trained predictive protein language NLP system.

In an aspect, the computer-implemented method further comprises:
receiving an input query, from a user interface device coupled to the executable program, comprising a candidate amino acid sequence;
generating, by the executable program, an output comprising a prediction including one or more biophysiochemical properties for the candidate amino acid sequence; and
displaying (optionally), the output on a display screen of a device, the predicted one or more biophysiochemical properties for the candidate amino acid sequence.

In an aspect, the computer-implemented method further comprises: receiving an executable program corresponding to the predictive protein language NLP system, the NLP system trained in the first phase by masking at least a portion of individual amino acids in the diversified protein sequence dataset. In aspects, about 5-25%, 10-20%, 12-17%, or 15% of the amino acids in the diversified protein sequence dataset are masked.

In another aspect, the computer-implemented method further comprises: receiving an executable program corresponding to the predictive protein language NLP system, the NLP system trained in the first phase using a diversified protein sequence dataset that has undergone individual amino acid-level tokenization, n-mer level tokenization, or sub-word level tokenization of respective protein sequences.

In another aspect, the computer-implemented method further comprises: receiving an executable program corresponding to a predictive protein language NLP system, the NLP system trained in the first phase using a diversified protein sequence dataset, wherein about 5-25%, 10-20%, 12-17%, or 15% of the amino acids in the diversified protein sequence dataset are masked.

In an aspect, the computer-implemented method further comprises: receiving an executable program corresponding to a predictive protein language NLP system, the NLP system generated by training in a first phase a first neural network and in a second phase a second neural network comprising features from the first neural network.

In another aspect, the first neural network comprises at least one first transformer model (e.g., a first transformer model and a second transformer model). In aspects, the first transformer model comprises at least one encoder and at least one decoder. In still other aspects, the first neural network comprises a second transformer model.

In still another aspect, the first transformer model comprises a transformer model with attention (e.g., self-attention). In still other aspects, the second transformer model comprises a transformer model with attention (e.g., self-attention).

In yet another aspect, the second neural network comprises for example, a perceptron or a second transformer model. In aspects, the second transformer model comprises at least one encoder and at least one decoder.

In still another aspect, the second transformer model comprises a transformer model with attention.

In another aspect, the transformer model with attention further comprises a robustly optimized bidirectional encoder representations from transformers model.

In still another aspect, the first and/or second neural network comprises a LSTM model.

In another aspect, the computer-implemented method further comprises: receiving a plurality of candidate amino acid sequences generated in silico, generating a prediction of whether the respective candidate amino acid sequences have one or more biophysiochemical properties. In yet another aspect, the candidate amino acid sequences are displayed according to a ranking of the one or more biophysiochemical properties.

In still another aspect, the one or more predicted biophysiochemical properties include but are not limited to: protein expression, binding affinity of a protein to a target, stability of a protein, fluorescence (log fluorescence intensity) of a protein, crystallization of a protein, therapeutic efficacy of a protein, biophysiochemical properties related to structure-function of the protein (e.g., cellular mechanisms, disease-causing functional changes, disease prevention, diagnosis, and treatment).

In yet another aspect, the computer-implemented method further comprises: providing for display on a display screen information from a salience module that indicates a contribution of respective amino acids to the prediction of the one or more biophysiochemical properties.

In still another aspect, the computer-implemented method further comprises: providing on the display screen information from a salience module that indicates a level of attention for each amino acid of the candidate sequence.

### System

In an embodiment, a system or apparatus is provided for training a predictive protein language NLP system comprising one or more processors to predict biophysiochemical properties of an amino acid sequence according to any of the methods provided herein.

A system or apparatus to predict biophysiochemical properties of an amino acid sequence comprising one or more processors for executing instructions corresponding to a predictive protein language NLP system to:
provide a predictive protein language NLP system trained to predict a biophysiochemical property;
receive an input query, from a user interface device coupled to the trained predictive protein language NLP system, comprising a candidate amino acid sequence;
generate, by the trained predictive protein language NLP system, a prediction including one or more biophysiochemical properties for the candidate amino acid sequence; and
display (optionally), on a display screen of a device, the predicted one or more biophysiochemical properties for the candidate amino acid sequence.

In aspects, a system or apparatus is provided to predict biophysiochemical properties of an amino acid sequence comprising one or more processors for executing instructions corresponding to a predictive protein language NLP system, the system:
trained, in a first phase, with a diversified protein sequence dataset in a self-supervised manner, and
trained, in a second phase and including features from the first phase. In other aspects, the second phase of training comprises training with an annotated protein sequence dataset in a supervised manner, wherein the annotated protein sequence dataset comprises individual protein sequences that are annotated with one or more biophysiochemical properties.

In still further embodiments, a system or apparatus is provided for executing instructions corresponding to a predictive protein language NLP system to predict biophysiochemical properties of an amino acid sequence according to the methods provided herein.

In another aspect, the system comprises a first neural network comprising a first transformer model. In aspects, the first transformer model comprises at least one encoder and at least one decoder. In still another aspect, the first transformer model comprises a transformer model with attention (e.g., self-attention). In another aspect, the system additionally comprises a first neural network comprising a second transformer model. In still another aspect, the second transformer model comprises a transformer model with attention (e.g., self-attention).

In yet another aspect, the system comprises a second neural network comprising for example, a perceptron or a transformer model. In aspects, the transformer model comprises at least one encoder and at least one decoder. In still another aspect, the transformer model comprises a transformer model with attention.

In another aspect, the transformer model with attention further comprises a robustly optimized bidirectional encoder representations from transformers model.

### Computer Readable Media

According to yet another embodiment, a computer program product is provided, the computer program product comprising a computer readable storage medium having instructions for training a predictive protein language NLP system to predict biophysiochemical properties of an amino acid sequence embodied therewith, the instructions executable by one or more processors to cause the processors to train the predictive protein language NLP system to predict the biophysiochemical properties of an amino acid sequence according to the methods provided herein.

According to still another embodiment, a computer program product for predicting biophysiochemical properties of an amino acid sequence is provided, the computer program product comprising a computer readable storage medium having instructions corresponding to a predictive protein language NLP system embodied therewith, the instructions executable by one or more processors to cause the processors to predict biophysiochemical properties of an amino acid sequence according to the methods provided herein.

In another aspect, the first neural network comprises a first transformer model. In aspects, the first transformer model comprises at least one encoder and at least one decoder.

In still another aspect, the first transformer model comprises a transformer model with attention (e.g., self-attention).

In another aspect, the first neural network comprises a second transformer model with attention (e.g., self-attention). In aspects, the second transformer model comprises a transformer model with attention (e.g., self-attention).

In yet another aspect, the second neural network comprises a perceptron or a transformer model. In aspects, the transformer model comprises at least one encoder and at least one decoder.

In still another aspect, the second neural network comprises for example, a perceptron or a transformer model with attention.

In another aspect, the transformer model with attention further comprises a robustly optimized bidirectional encoder representations from transformers model (RoBERTa).

In still other aspects, a computer-readable data carrier is provided having stored thereon the computer program product for predicting biophysiochemical properties according to any of the methods or systems provided herein.

In another aspect, a computer-readable storage medium is provided having stored thereon the computer program product for predicting biophysiochemical properties according to any of the methods or systems provided herein.

In other aspects, a system is provided comprising one or more processors and the computer readable storage medium/computer program product for predicting biophysiochemical properties according to any of the methods or systems provided herein.

In aspects, the predictive protein language NLP system may receive two or more candidate amino acid sequences.

In aspects, the first neural network may comprise at least two transformers, and the embeddings from the first transformer and the second transformer may be provided to the second neural network.

In still other aspects, a computer-implemented method, system and/or computer program product for predicting biophysiochemical properties of amino acid sequences using natural language processing (NLP) is provided, comprising:
in a first phase, training a first neural network comprising a first transformer with tokenized, masked epitope sequences, and a second transformer with tokenized masked TCR sequences, optionally in a self-supervised manner; and
in a second phase, training a second neural network, to predict TCR-epitope binding, wherein the second neural network comprises features or embeddings from the first phase of training.

In aspects, various inputs (e.g., categorical variable and embeddings) are provided as input (e.g., categorical variables) to fully connected layers of a neural network (e.g., such as a perceptron).

In other aspects, the following are treated as embedded variables (by tokenizing on an individual amino acid level): epitope, TCR-A-CDR3, and TCR-B-CDR3 sequences. In aspects, the input to the language model for the second phase comprises concatenated representations of sequence and categorical feature embeddings (e.g., gene, family variables). In aspects, the following are treated as categorical variables: TRA-v-gene, TRA-v-family, TRB-v-gene, TRB-v-family, TRB-d-gene, TRB-d-family, TRA-j-gene, TRA-j-family, TRB-j-gene, TRB-j-family, MHCa_HLA_protein, and HCa_allele).

The summary is not intended to restrict the disclosure to the aforementioned embodiments. Other aspects and iterations of the disclosure are provided below.

### BRIEF DESCRIPTION OF THE DRAWINGS

Generally, like reference numerals in the various figures are utilized to designate like components.

FIG. 1 is an illustration of an example computing environment for the protein language natural language processing (NLP) system in accordance with certain aspects of the present disclosure.

FIG. 2 is a block diagram of the protein language NLP system of FIG. 1 in accordance with certain aspects of the present disclosure.

FIG. 3A is an illustration of individual amino acid-level tokenization of a protein sequence in accordance with certain aspects of the present disclosure.

FIG. 3B is an illustration of n-mer tokenization of a protein sequence in accordance with certain aspects of the present disclosure.

FIG. 3C is an illustration of sub-word tokenization of a protein sequence in accordance with certain aspects of the present disclosure.

FIG. 4 is an illustration of randomly masking a tokenized protein sequence in accordance with certain aspects of the present disclosure.

FIG. 5A is a flow diagram showing generation of a training dataset for a first neural network in accordance with certain aspects of the present disclosure.

FIG. 5B is a flow diagram showing training of a first neural network with a first training dataset in accordance with certain aspects of the present disclosure.

FIG. 5C is a flow diagram showing training of a second neural network subjected to transfer learning with a second dataset in accordance with certain aspects of the present disclosure.

FIG. 5D is a flow diagram showing updating a trained second neural network with experimental data in accordance with certain aspects of the present disclosure.

FIG. 6 is an illustration of generating prediction probabilities for each masked instance of an amino acid by the first neural network trained on randomly masked, tokenized protein sequences in accordance with certain aspects of the present disclosure.

FIG. 7 is an illustration of transfer learning, in accordance with certain aspects of the present disclosure.

FIG. 8A is an example architecture of the first neural network, in accordance with certain aspects of the present disclosure.

FIG. 8B is an example architecture of the second neural network, in accordance with certain aspects of the present disclosure.

FIG. 9A is a high-level flowchart of operations of training a protein language NLP system, in accordance with certain aspects of the present disclosure.

FIG. 9B is another flowchart of example operations for training a predictive protein language NLP system comprising a transformer that predicts a binding affinity of an antigen to a TCR, in accordance with the embodiments provided herein.

FIG. 10 is a high-level flowchart of operations of an executable corresponding to the trained protein language NLP system, in accordance with certain aspects of the present disclosure.

FIG. 11A is a high-level flowchart of operations of the trained protein language NLP system, in accordance with certain aspects of the present disclosure.

FIG. 11B is another flowchart of example operations for accessing a trained predictive protein language NLP system comprising a transformer that predicts a binding affinity of an epitope to a TCR, in accordance with the embodiments provided herein.

FIG. 12A is a screenshot showing aspects of a portion of a user interface in accordance with certain aspects of the present disclosure.

FIG. 12B is another screenshot of a portion of a user interface showing an enlarged view of classification results in accordance with certain aspects of the present disclosure.

FIG. 12C is another screenshot of a portion of a user interface showing an enlarged view of a salience module in accordance with certain aspects of the present disclosure.

FIG. 12D is another screenshot showing a portion of a user interface with layers of attention in accordance with certain aspects of the present disclosure.

FIG. 13 is a block diagram of an example computing device, in accordance with certain aspects of the present disclosure.

FIG. 14 is a bar graph showing performance aspects of the protein language NLP (PL-NLP) system for predicting protein fluorescence and protein stability as compared to a benchmark in accordance with certain aspects of the present disclosure.

FIG. 15 is a bar graph showing performance aspects of the protein language NLP system for predicting protein expression and protein crystallization as compared to a benchmark in accordance with certain aspects of the present disclosure.

FIG. 16A is a bar graph showing performance aspects of the protein language NLP system for predicting binding as compared to benchmarks (Net-TCR and ERGO) in accordance with certain aspects of the present disclosure.

FIG. 16B is another bar graph showing performance aspects of the protein language NLP system for predicting binding affinity as compared to a benchmark (TCellMatch) in accordance with certain aspects of the present disclosure.

FIG. 17 is a diagrammatic illustration of a model architecture used for TCR-epitope binding affinity in accordance with certain aspects of the present disclosure.

FIG. 18A shows categories of predicted TCR-binding specificity by the protein language NLP system in accordance with certain aspects of the present disclosure.

FIG. 18B shows results of predicted TCR-binding affinity for a plurality of epitopes by the protein language NLP system in accordance with certain aspects of the present disclosure.

FIG. 19 is an illustration showing various example datasets, neural network models, and tokenization schemes suitable for the protein language NLP system in accordance with certain aspects of the present disclosure.

FIG. 20 shows a comparison of the protein language NLP system to other machine learning approaches, in accordance with certain aspects of the present disclosure.

### DETAILED DESCRIPTION

Amino acids are the building blocks from which a variety of macromolecules are formed, including peptides, proteins, and antibodies. These macromolecules play pivotal roles in a variety of cellular processes, for example, by forming enzyme complexes, acting as messengers for signal transduction, maintaining physical structures of cells, and regulating immunological responses. For example, enzyme complexes catalyze biochemical reactions, messengers act in various signal transduction pathways to regulate and control cellular processes, scaffold and support proteins provide shape and mechanical support to cells, and antibodies provide an immune system defense against viruses and bacteria.

Amino acids have an amino group (N-terminus), a carboxyl group (C-terminus), and an R group (a side chain) that confers various properties (e.g., polar, nonpolar, acidic, basic, etc.) to the amino acid. Amino acids may form chains through peptide bonds, which is a chemical bond formed by joining the C-terminus of an amino acid with the N-terminus of another amino acid. There are twenty naturally occurring amino acids, and unnaturally occurring amino acids have been synthesized as well. The amino acid side chains are thought to influence protein folding and shape.

The overall shape of a protein may be described at various levels, including primary, secondary, tertiary, and quaternary structures. The sequence or order of amino acids in a protein corresponds to its primary structure, also referred to as a protein backbone. Secondary structures such as alpha helices, beta sheets, turns, coils or other structures may form locally along the protein backbone. A three-dimensional shape of the protein structure/subunit, which includes local secondary structures, forms a tertiary structure, and quaternary structures are formed from the association of multiple protein structures/subunits. Thus, the structure of proteins may be described at various levels. Proteins range in size from tens to thousands of amino acids, with many proteins on the order of about 300 amino acids.

The sequence of amino acids confers structure and protein function. A protein may have one or more domains (e.g., a local three-dimensional fold relative to the full protein structure) corresponding to a particular function and such domains may be evolutionarily conserved. Thus, evolutionary demands, at least in part, are thought to influence protein sequences, leading to conservation of amino acids at positions that govern protein folding and/or function.

The natural language processing approaches provided herein, a subfield of artificial intelligence (Ai)/machine learning (ML), extend beyond traditional one-shot predictive modelling approaches in which an Ai/ML model is trained on a particular, usually narrow, topic. Instead, present approaches train a protein language NLP system on a diverse dataset or library of protein sequences. The trained protein language NLP system may be further refined to predict biophysiochemical properties.

By self-supervised learning, it is meant that supervision is not needed, as the data itself (e.g., next amino acid) provides supervision. By supervised learning, it is meant that annotated/labeled data is provided so that the system learns to map an input to an output based on example input output pairs.

Natural language processing refers to a subfield of artificial intelligence geared towards processing of text-based input. Artificial neural networks may be utilized for natural language processing techniques. In the present application, NLP techniques are applied to protein sequences.

The protein language NLP system may be customized to a variety of applications, including crystallization, binding, antibody optimization, protein expression, protein stability, TCR-epitope binding affinity, microbiome analysis, enzyme engineering, etc. Present techniques may further be applied to a wide variety of applications in vaccine development including protein-related aspects of this process, such as solubility, productivity, aggregation, homogeneity, integrity, structural stability, protein-protein interactions, TCR- and BCR-epitope recognition, etc. These techniques may be used to prioritize vaccine antigens based upon a categorization and/or a ranking provided by the protein language NLP system. Present techniques may also be used to generate and explore binding predictions for novel TCR and epitope sequences as well as interrogate and visualize which amino acid residues contribute to the prediction. Additionally, the protein language NLP system, after a first phase of training, may be used as an off-the-shelf product that is customized (fine-tuned) for a particular application.

In aspects, a computer-implemented method for predicting biophysiochemical properties of an amino acid sequence using natural language processing (NLP) is provided. In aspects, computer implemented methods for predicting binding or binding affinity are provided for TCRs and their epitopes.

In aspects, the protein language NLP system includes at least one transformer model (e.g., one transformer, two transformers, etc.). Each transformer model includes a transformer model with attention (e.g., self-attention). The transformer model is a robustly optimized bidirectional encoder representation with transformers.

Advantages of present techniques include tailoring a diversely trained neural network to any of a number of specific applications. By diversely training a first neural network during a first phase, the system learns the lexicography of protein sequences. The knowledge gained from training the first neural network in the first phase with a diverse and large dataset may be transferred to the second neural network and fine-tuned for a specific application. Fine-tuning may be performed with an annotated, compact dataset that is smaller than the diverse dataset (e.g., by a factor of 2, a factor of 5, a factor of 10, a factor of 20, etc.), and the second phase of training may be performed more quickly than the first phase of training. These approaches provide an alternative to time consuming, one-shot machine learning approaches that need large amounts of annotated data, while offering accelerated development of machine learning applications.

In addition to offering reduced computational time as compared to other approaches, present techniques do not rely on ingesting large volumes of data that may be difficult to obtain, such as atomic data needed by molecular simulation techniques. Instead, present approaches first train on a diverse dataset of amino acid sequences in an unsupervised/self-supervised manner to learn the rules of protein sequences and structure, and then apply this knowledge in a second phase of training to tailor the system to a specific application.

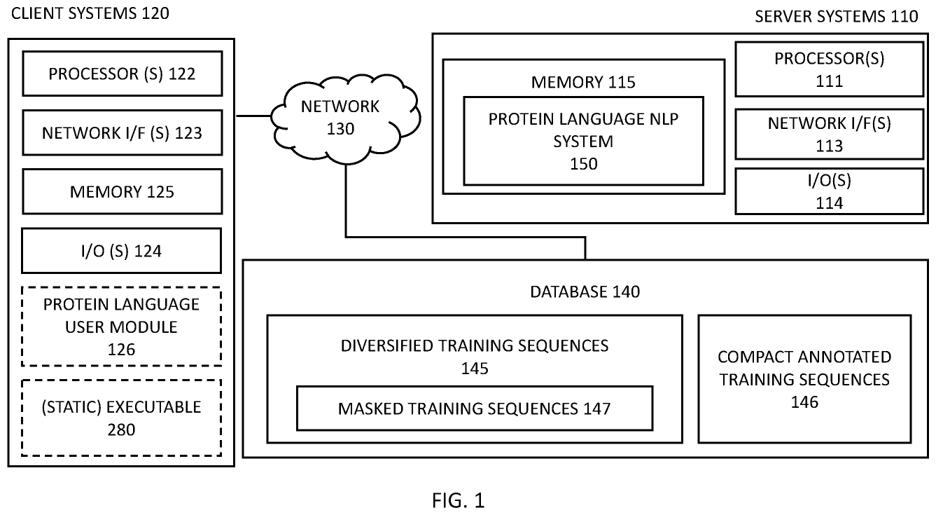

Accordingly, by analyzing amino acid sequences with natural language processing techniques, a trained lexicographic protein language system may be generated based on a conserved “language” of proteins that may quickly and easily be fine-tuned to a variety of specific applications. Surprisingly, present approaches apply natural language processing techniques (e.g., neural nets such as transformers and transfer learning) to the biological domain, offering improved predictive capabilities of various biophysiochemical properties that meet or exceed current benchmarks. With reference now to FIGS. 1-20, examples of a computer-implemented method, a computer-implemented system, a computer program product and results are provided.

FIG. 1 shows an example computing environment for use with the predictive protein language NLP system provided herein. The computing environment may include one or more server systems 110 and one or more client/end-user systems 120. Server systems 110 may communicate remotely with client systems 120 over a network 130 with any suitable communication medium (e.g., via a wide area network (WAN), a local area network (LAN), the Internet, an Intranet, or any other suitable communication medium, hardwire, a wireless link, etc.). Server systems 110 may comprise a predictive protein language NLP system 150, stored in memory 115 that is trained on protein sequences stored in database 140.

Server systems 110 may comprise a computer system equipped with one or more processor(s) 111 (e.g., CPUs, GPUs, etc.), one or more memories 115, internal or external network interfaces (I/F) 113 (e.g., including but not limited to a modem, a network card, etc.), and input/output (I/O) interface(s) 114 (e.g., a graphical user interface (GUI) or other interface (e.g., command line prompts, menu screens, etc.) to receive input from an input device (e.g., a keyboard, a mouse, etc.) or to display output on a display screen. The server system may comprise any commercially available software (e.g., server operating system, server/client communications software, browser/interface software, device drivers, etc.) as well as custom software (e.g., protein language NLP system 150, etc.). In some aspects, server systems 110 may be, for example, a server, a supercomputer, a distributed computing platform, etc. Server systems 110 may execute one or more applications, such as software for the predictive protein language NLP system 150 that predicts biophysiochemical properties of candidate sequence(s).

Memory 115 stores program instructions that provide the functionality for the predictive protein language NLP system 150. These program instructions are generally executed by processor(s) 111, alone or in combination with other processors.

Client systems 120 may comprise a computer system equipped with one or more processor(s) 122, one or more memories 125, internal or external network interface(s) (I/F) 123 (e.g., including but not limited to a modem, a network card, etc.), input/output (I/O) interface(s) 124 (e.g., a graphical user interface (GUI) or other interface (e.g., command line prompts, menu screens, etc.) to receive input from an input device (e.g., a keyboard, a mouse, etc.) by a user or to display output on a display screen. The client system 120 may comprise any commercially available software (e.g., operating system, server/client communications software, browser/interface software, etc.) as well as any custom software (e.g., protein language user module 126, etc.). In some aspects, client systems 120 may be, for example, any suitable computing device, such as a desktop computing device, a laptop computing device, a tablet computing device, a mobile phone computing device, etc.

Client systems 120 may execute via one or more processors one or more applications, such as software corresponding to the protein language user module 126. In some aspects, protein language user module 126 may enable a user to provide candidate protein sequence(s) to the protein language NLP system and to receive predictions of biophysiochemical properties for candidate protein sequences(s). In aspects, client systems 120 may provide one or more candidate protein sequence(s) to server systems 110 for analysis by protein language NLP system 150, and protein language NLP system may analyze the candidate sequence(s) to return one or more biophysiochemical properties predicted by the system. Thus, in aspects, client systems 120 may access server systems 110, which hosts/provides the trained predictive protein language NLP system.

In aspects, the trained protein language NLP system 150 continues to undergo additional training as new data is available. In this dynamic mode of operation, the protein language NLP system continues to be trained at the server side, and a client system may access the protein language NLP system through network 130 via protein language user module 126.

In an alternative embodiment, once the protein language NLP system has been trained (e.g., with a first phase of training on a diversified dataset and a second phase of training with a specific annotated dataset), the trained protein language NLP system may be converted into an executable for execution on client systems 120, allowing analysis of candidate protein sequence(s) to proceed in a stand-alone mode of operation.

In this stand-alone mode of operation, the client system runs an executable 280 corresponding to the trained predictive protein language NLP system in a static mode of operation. In this mode, the static executable 280 corresponding to the predictive protein language NLP system 150 does not undergo additional training and is locked in a static configuration. In operation, the executable may receive and analyze candidate sequence(s) to return one or more predicted biophysiochemical properties for the candidate sequence(s).

Typically, the client device includes protein language user module 126 or executable 280.

Thus, in aspects, protein language NLP system 150 may be compiled into an easy-to-use package/executable (e.g., python, etc.). In other aspects, protein language NLP system 150 may be provided as a software as a service (“SaaS”), in which a user remotely accesses the protein language NLP system 150 (trained) hosted by a server.

The environment of present invention embodiments may include any number of computers or other processing systems (e.g., client/end-user systems, server systems, etc.) and databases or other storage repositories arranged in any suitable fashion. These embodiments are compatible with any suitable computing environment (e.g., distributed, cloud, client-server, server-farm, network, mainframe, stand-alone, etc.).

A database 140 may store various data (e.g., diversified training sequences 145 for diversified training in phase one, and annotated, compact training sequences 146 for specific applications in phase two). In some aspects, a diversified dataset may comprise diversified training sequences from a variety of species and/or from a class/genus, e.g., of receptors, of epitopes, etc. In some aspects, masked training sequences may be stored in database 140 as masked training sequences 147. In some aspects, the database may be implemented by any conventional or other suitable database or storage system, and may be local to or remote from server systems 110 and client systems 120. In aspects, the database may be connected to a network 130 and may communicate with the client and/or the server via any appropriate local or remote communication medium (e.g., local area network (LAN), wide area network (WAN), Internet, Intranet, hardwire, wireless link, cellular, satellite, etc.).

The system may employ any number of any conventional or other databases, data stores or storage structures (e.g., files, databases, data structures, data or other repositories, etc.) to store information (e.g., training sequences, candidate sequences, biophysiochemical property predictions, neural network features such as neural network configurations and/or hyperparameters, embeddings, etc.). The database system may be included within or coupled to the server and/or client systems. The database systems and/or storage structures may be remote from or local to the server or other processing systems, and may store any desired data.

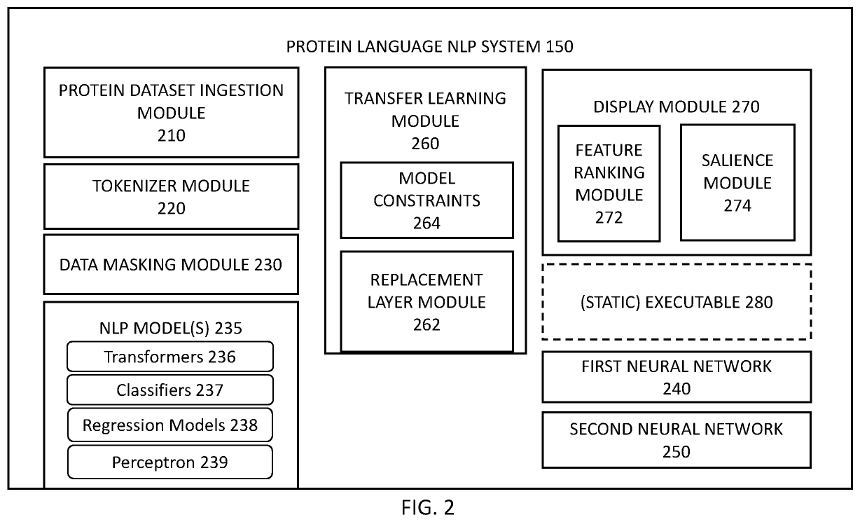

As shown in FIG. 2, the protein language NLP system 150 comprises a protein dataset ingestion module 210, a tokenizer module 220, a data masking module 230, a first neural network 240 (an artificial neural network), a second neural network 250 (an artificial neural network), a transfer learning module 260, and a display module 270. In some aspects, the protein language NLP system 150 may also include an executable 280 configured to run on client systems/devices. The executable 280, which corresponds to the protein language NLP system 150, does not undergo further learning, but rather, is compiled as a static configuration. It is to be understood that the neural networks provided herein refer to artificial neural networks.

The protein language NLP system 150 comprises a first neural network 240 and a second neural network 250 that is trained in two phases. In some aspects, the first neural network 240 is trained in the first phase on a diverse set of protein sequences from a repository that contains compilations of proteins with different functions (e.g., structural, neurological, transport, mechanical, calcium/sodium channels, signal transduction, growth and development, etc.). By training on a diverse set of protein sequences, the protein language NLP system learns, in an unsupervised/self-supervised manner, “rules” of protein sequences. An annotated dataset is not needed as the next amino acid is known (except for end of sequence). Once the first phase of training is complete, transfer learning module 260 transfers knowledge from training the first neural network to a second neural network 250. The second neural network is then retrained (fine-tuned) for a specific biological application using an annotated, compact dataset that is of a reduced size as compared to the diverse dataset.

In other aspects, the first neural network 240 may comprise two transformers (e.g., self-attention). The first transformer may be trained on masked, tokenized epitope sequences and the second transformer may be trained on categories of TCR sequences/TCRs. Information (e.g., in the form of embeddings, categorical variables, sequences, concatenated representations of sequence and categorical feature embeddings, or combinations thereof) may be provided to the second neural network from the first neural network, and the second neural network (e.g., a perceptron) may undergo further refinement as provided herein.

Thus, the first neural network refers to a neural network trained in a first phase, and the second neural network refers to another neural network trained in a second phase.

NLP model 235 may contain any suitable machine learning model for the embodiments provided herein. Models may be constructed and may comprise algorithms including but not limited to neural networks, deep learning networks, generative networks, convolutional neural networks, long short term memory networks, transformers, transformers with attention, robustly optimized neural networks, etc. In aspects, a robustly optimized bidirectional encoder representations from transformers (RoBERTa) is provided (see, e.g., Liu et al., RoBERTa: A Robustly Optimized BERT Pretraining Approach, (2019), arXiv.org/abs/1907.11692). In aspects, the NLP model 235 may comprise any suitable transformer(s) 236, classifier(s) 237, regression model(s) 238, perceptron 239, etc. In aspects, a classifier(s) 237 may predict a label or category of a given input value (e.g., binding, not binding). In aspects, a regression model(s) 238 may predict a discrete value. Ranges may map discrete values to particular properties (e.g., a first range to indicate strong binding, a second range to indicate medium binding, and a third range to indicate weak binding).

Protein dataset ingestion module 210 ingests datasets from public and/or private repositories. In some aspects, the protein dataset ingestion module may ingest protein sequences downloaded from a database, and may format the dataset (e.g., by removing extraneous text and/or extracting the protein sequences from database records) in order to provide this data as input to the tokenizer module 220. Publicly and/or privately available sequence data may be provided in any suitable format (e.g., FASTA, SAM, etc.) from any suitable source, e.g., UniProt (uniprot.org), EMBL (ebi.ac.uk), and/or PFam (pfam.xfam.org, etc.). In various implementations, the protein sequence may be parsed and stored in one or more data structures, including but not limited to trees, graphs, lists (e.g., linked list), arrays, matrices, vectors, and so forth.

In some aspects, the protein dataset ingestion module may remove duplicate sequences obtained from ingesting multiple overlapping protein repositories. By identifying and removing duplicates, overrepresentation of a given protein may be minimized.

In aspects, the protein dataset ingestion module may receive as input protein sequence information downloaded from a publicly available database, e.g., in FASTA, XML, or another text based format. For example, individual records of a bulk database download may contain one or more fields including a record identifier (e.g., a numeric and/or text identifier such as one or more NCBI identifiers including a library accession number, protein name, etc.) as well as the protein sequence itself (amino acid sequence). The protein dataset ingestion module may parse each downloaded record/entry obtained from the database (e.g., in a FASTA, XML, text, or other format etc.) into a data structure or structured data (e.g., a format such as an array or matrix) suitable for input into the tokenization module 220. For example, the output of the protein dataset ingestion module may comprise a data structure or structured data with each entry including an identifier and the corresponding sequence listing (e.g., an array or matrix of entries).

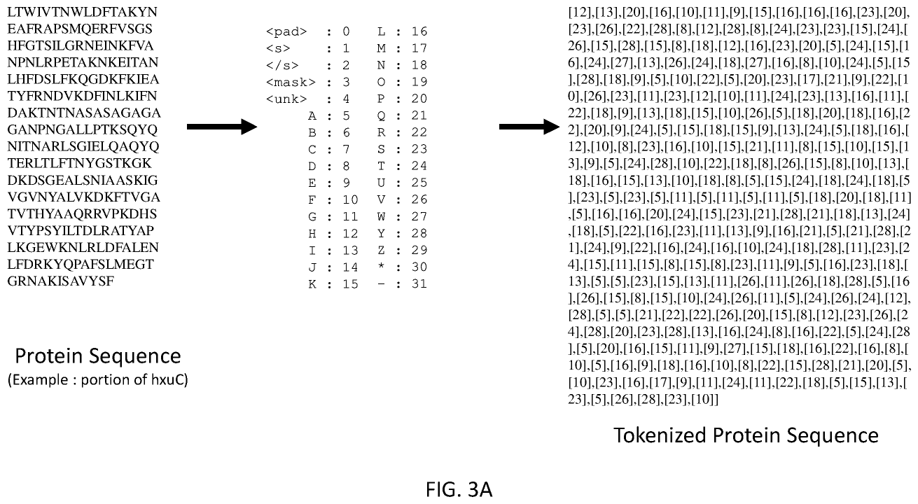

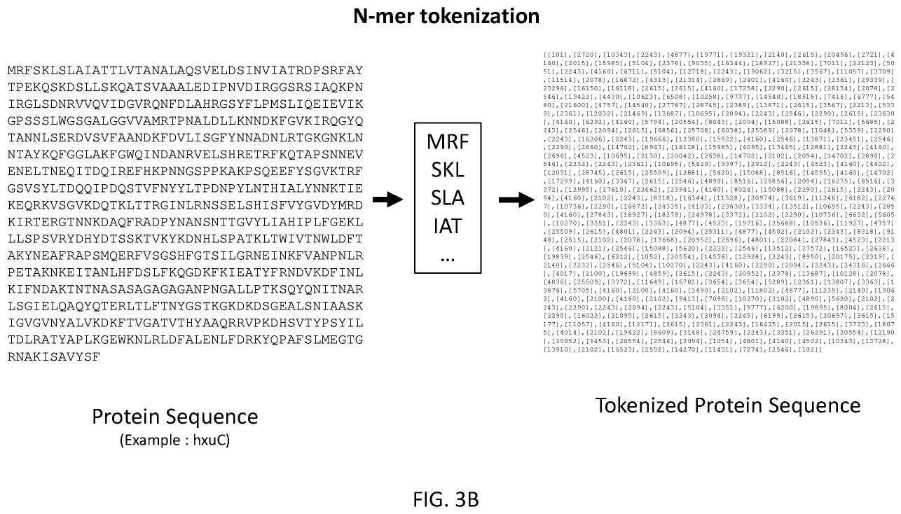

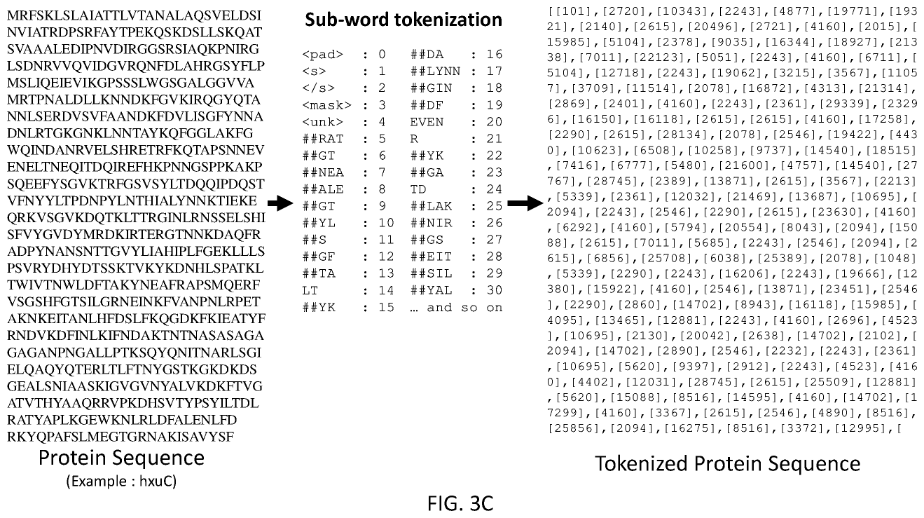

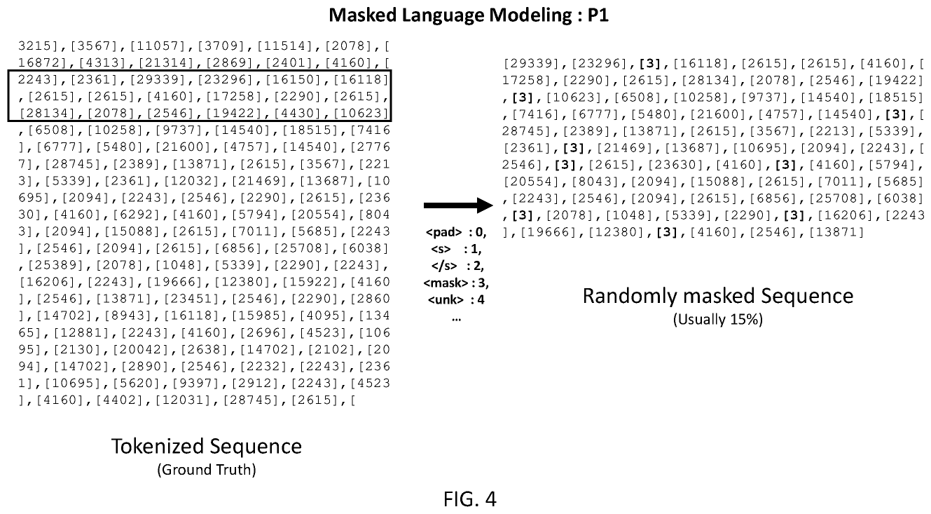

Tokenizer module 220 performs tokenization on the amino acid sequences. Tokenizer module 220 receives the output of the ingestion module (e.g., comprising a data structure or structured data with each entry of an array comprising an identifier and a corresponding amino acid sequence), and converts the received data structure or structured data into a tokenized data structure. Tokenization of an amino acid sequence comprises parsing the amino acid sequence into components (e.g., individual amino acids, n-mers, sub-words, etc.) and mapping each component to a value. For example, an amino acid sequence may be separated into individual amino acids, and each individual amino acid may be mapped to a numeric value (e.g., MRF . . . ->[17],[22],[10] . . . ). The output of the tokenizer module may be another data structure or structured data (e.g., comprising an array or matrix structure) with each entry corresponding to a tokenized representation of an amino acid sequence. In aspects, inputs may be embedded into the system via individual amino acid tokenization. (Other inputs may be treated as categorical variables that may not be subject to individual amino acid tokenization). In some aspects, the components are mapped to numeric values to streamline processing. Various approaches for tokenization and masking that may be utilized in accordance with training the protein language NLP system are provided below with reference to for example, FIGS. 3A-3C and FIG. 4.

With reference to FIG. 3A, in some aspects, tokenization may be performed at the individual amino acid-level (individual amino acid-based tokenization), also referred to as single or individual amino acid-level tokenization, in which each individual amino acid is mapped to a different numeric value. For example, the amino acid for alanine “A” may be mapped to a numeric value “5,” the amino acid for valine “V” may be mapped to a numeric value “26”, and so forth. Other characters such as various types of whitespace characters (e.g., padding, space, tab, return, etc.), unknown characters, wildcard characters, hyphens, etc. may each be mapped to other numeric values. An example of an individual amino acid-level tokenization scheme is provided in FIG. 3A.

With reference to FIG. 3B, in another aspect, n-mer tokenization may be performed. In this approach, short n-mers of adjacent amino acids (e.g., where n is a numeric value such as 2, 3, 4 or more to form respective strings of two amino acids, three amino acids, four amino acids, etc.), are each mapped to numeric values. Examples of n-mers include but are not limited to: two-mers such as AV, LK, CY, NW, etc., three-mers such as ASK, SKJ, VAL, TGW, JHS, etc. and so forth.

Referring to FIG. 3C, in another aspect, sub-word tokenization may be performed. In this approach, sub-words or strings of amino acids of varying length are each mapped to particular numeric values. Examples of sub-words include but are not limited to: ##RAT, ##GT, R, TD, ##LYNN, etc. In some aspects, sub-words are determined based upon analysis of protein sequences or may be based upon knowledge from the literature and/or subject matter experts.

Sequences may be tokenized according to any suitable tokenization scheme provided herein.

Once tokenized, one or more sequences may be subject to masking, which hides the identity of amino acids at random locations in the amino acid sequences. With reference to FIG. 2, data masking module 230 may mask amino acid sequences, obscuring the identity of amino acids at random locations to create a training dataset (e.g., masked training sequences 147) for the first neural network 240. Data masking module may receive as input, the output of the tokenizer module (e.g., a data structure or structured data comprising an array or matrix structure) with each entry comprising a tokenized representation of an amino acid sequence. The data masking module may utilize a masking function that randomly selects amino acids (e.g., a percentage of amino acids within a protein sequence) and masks the identity of these amino acids. Masking hides the identity of amino acids at particular locations in the sequence. For example, an amino acid at a given position may be known to be a valine, and the data masking module hides or obfuscates the identity of the amino acid at this position by replacing it with a designated masking value. The output of the data masking module may comprise another data structure or structured data, for example, comprising an array or matrix of entries, with each entry including a masked and tokenized amino acid sequence, which is provided as input to a first neural network for a first phase of training.

For example, and with reference to FIG. 4, a tokenized sequence in which individual amino acids are represented as numeric values is masked, with the masked amino acids represented by a designated masking value (e.g., in this case, <3>). In some aspects, about 5%, 6%, 7%, 8%, 9%, 10%, 11%, 12%, 13%, 14%, 15%, 16%, 17%, 18%, 19%, 20%, 21%, 22%, 23%, 24%, 25% of amino acid sequences, collectively across the library of proteins or individually with respect to particular proteins, may be masked. For example, masking may be applied to an individual protein such that 15% of the respective protein is masked (e.g., with shorter sequences having fewer absolute numbers of masked amino acids than longer sequences) or masking may be applied across the library (without regard to individual proteins) such that 15% of the total number of amino acids in the library are masked.

In some aspects, between 5-25% of the protein sequence, between 10-20%, between 11-19%, between 12-18%, between 13-17%, between 14-16%, or about 15% of the protein sequence is masked.

In some aspects, amino acids are masked in a random manner In still other aspects, masking may be constrained such that the masked amino acids are not permitted to be adjacent to each other. For example, the masked amino acids may be separated by a minimum spacing such that there is 1, 2, 3, 4, 5, 6, 7, 8, 9, 10, 11, 12, 13, 14, 15, etc. unmasked amino acids between masked amino acids.

Referring back to FIG. 2, a first neural network 240 is provided for training with the masked, tokenized, diversified protein dataset (e.g., masked training sequences 147). The first neural network 240 may comprise any suitable NLP model 235 including but not limited to deep learning models designed to handle sequential data. In aspects, the first neural network may comprise a transformer model, a generative model, a LSTM model, a shallow neural network model, etc.

The first neural network 240 may be trained on one or more diversified dataset(s) (e.g., publicly available, privately available, or a combination thereof) such as UniProt (uniprot.org), EMBL (ebi.ac.uk), PFam (pfam.xfam.org), or any combination thereof and with or without preprocessing in a self-supervised manner Self-supervised training may proceed until meeting suitable criteria, for example, as specified by an AUC-ROC curve. For example, training may continue until reaching an AUC value of 0.7, 0.75, 0.8, 0.85, 0.90, 0.95, 0.96, 0.97, etc. An annotated dataset is not needed for training the first neural network, since the next amino acid is known (except for the end of the amino acid sequence).

In some aspects, transformer models, which are suitable for understanding context and long-range dependencies during self-supervised learning on large datasets, were shown to outperform other types of neural networks.

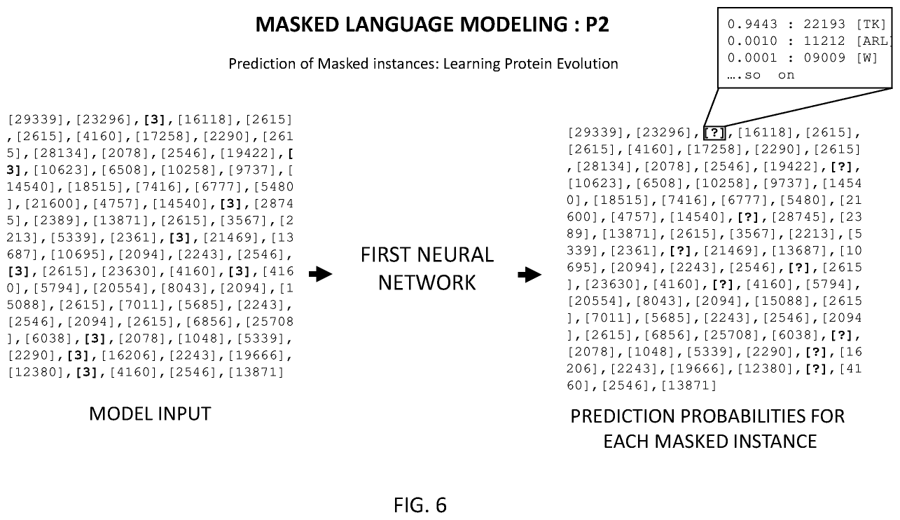

In some aspects, an attention-based transformer may be trained on masked, tokenized protein sequences, wherein the protein sequences have been masked to obscure about 15% of the amino acid identities. During training, the first neural network makes a determination of the masked value based upon a statistical likelihood. As the identity of the amino acid (true value) is known, the predicted amino acid identity may be compared to the true value, and this information may be provided back to the first neural network to improve the predictive ability of the first neural network. The output of the attention-based transformer model corresponds to another data structure or structured data, e.g., comprising entries of masked, tokenized amino acid sequences, with each masked amino acid instance associated with a probability of a specific amino acid at the masked position (see, FIG. 6).

Accordingly, in this first training step, the datasets contain a diversified listing of protein sequences that include proteins with different functions and/or from different classes. In aspects, the first neural network is trained broadly on a “language of proteins” without being specialized or biased towards one particular class or function. In some aspects, the protein language model may be trained on sequences limited to human origin or mammalian origin.

In other aspects, the first neural network 240 comprises a first transformer and a second transformer. The first transformer may be trained on TCR sequences, and the second transformer may be trained on epitopes. In aspects, the transformer may be a self-attention transformer (e.g., RoBERTa, etc.)

Once the first neural network 240 has been trained, the knowledge (e.g., neural network configuration, hyperparameters, layers, embeddings, etc.) obtained from this process may be transferred to a second neural network 250 (concatenated representations of sequence and categorical feature embeddings), and the second neural network may be fine-tuned through further training.

In aspects, the second neural network 250 may comprise any suitable NLP model 235 including but not limited to deep learning models designed to handle sequential data. For example, the second neural network may comprise a transformer model, a generative model, a LSTM model, a shallow neural network model, a fully connected neural network, a perceptron, etc. In aspects, the output layer of the second neural network may comprise a classifier, a (multi-output) regression model, that predicts a desired biophysiochemical property.

In aspects, the second neural network may comprise, for example, a perceptron, or a transformer model. In still further aspects, the transformer model may be a transformer model with attention. In still other aspects, the second neural network may be a perceptron.

In some aspects, transformer models, which are suitable for understanding context and long-range dependencies during self-supervised learning on large datasets, were shown to outperform other types of neural networks.

With respect to the present application, the first neural network refers to the neural network which is trained in a first phase on a dataset (e.g., a diverse set of protein sequences, a set of protein sequences of a particular category (e.g., TCR sequences, epitope sequences, etc.)), and the second neural network refers to a neural network containing information from the first phase of training and trained in a second phase on an annotated, compact, specific dataset as compared to the diversified dataset. Any suitable technique in transfer learning may be used to transfer knowledge from the first neural network to the second neural network. In still other aspects, the second neural network may be a perceptron.

Thus, a second neural network may refer to a neural network comprising information from the first neural network using any suitable approach (e.g., loading, copying, transferring embeddings/layers, custom generated data functions/variables, etc.) that is trained in a second phase for a particular application. For example, hyperparameters (e.g., weights, layers, labels, inputs, outputs, embeddings, variables, etc.) obtained from the trained first neural network may be transferred or loaded into a second neural network. In other aspects, processing may continue using a modified form of the first neural network (with a copy of the fully trained first neural network stored in memory for other applications).

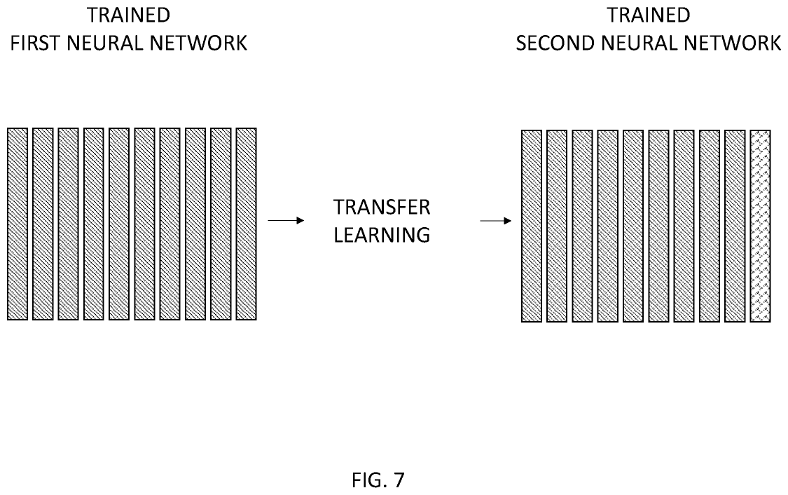

As discussed further below, the second neural network may optionally be modified prior to fine-tuning. These changes may include replacing one or more output layers of the trained first neural network with replacement layers via replacement layer module 262, such that the resulting neural network comprises layers (retained layers) from the first phase of training and replacement layers (see also, FIG. 7) that replace one or more output layers. In some aspects, model constraints 264 are applied to the retained layers to prevent or constrain modification of the weights of these layers during subsequent training with an annotated dataset, which tailors or fine-tunes the second neural network for a specific task. Model constraints 264 ensure that information from the first phase of training is retained by reducing/minimizing parameter changes for the retained layers. In some aspects, one or more retained layers may be released in a layer-by-layer manner as training progresses to allow small scale parameter modification.

In other aspects, the output of the first and second transformer may be transferred to a second neural network in any suitable manner, for example, in the form of information/variables (such as embeddings, categorical variables, hyperparameters, or combinations thereof), layers, etc. In other aspects, data may be transferred by obtaining embeddings from the first phase of training and providing the embeddings (in any suitable form) to the second neural network for the second phase of training Thus, the second training phase fine-tunes the second neural network to a specific application (e.g., binding affinity, crystallization, solubility, etc.). Specific applications include any biophysiochemical property, including any biological, chemical, or physical property influenced by protein sequence. Training may proceed until a specified AUC-ROC parameter has been met. In some aspects, the output layer of the second neural network determines whether a physiological feature is present (e.g., the output layer may act as a classifier regarding presence of a biophysiochemical feature such as expression or no expression, binding or no binding, etc.). In some aspects, the output layer comprises a measure of the specified feature, for example, a measure of binding affinity (e.g., high, medium, low, etc.). This approach may be extended to predict a variety of biophysiochemical properties as discussed further below.

Display module 270 provides various displays for users to visualize and explore the output of the protein language NLP system. For example, display module 270 comprises a salience module 274 (interpretability module), which provides interpretability into the protein language NLP system, providing users with insights into predictions at the amino acid level. Using this salience module, a user may gain insight into which amino acids of the protein sequence contributes to a specific biophysiochemical property (e.g., TCR-epitope interactions). In some aspects, the contributory amino acids are individually highlighted according to a color schema (e.g., red to blue, light to dark, etc.) in the display.

In other aspects, feature ranking module 272 may rank the output of the second neural network for a plurality of candidate amino acid sequences, based on any suitable parameter, e.g., strength of binding affinity, etc.

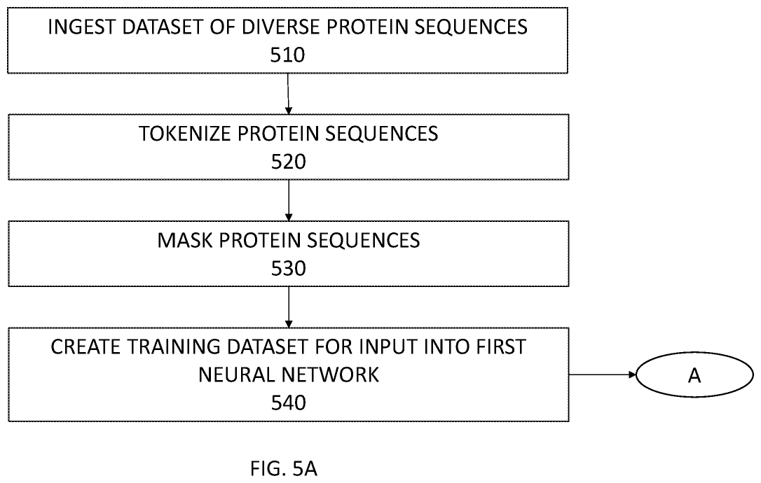

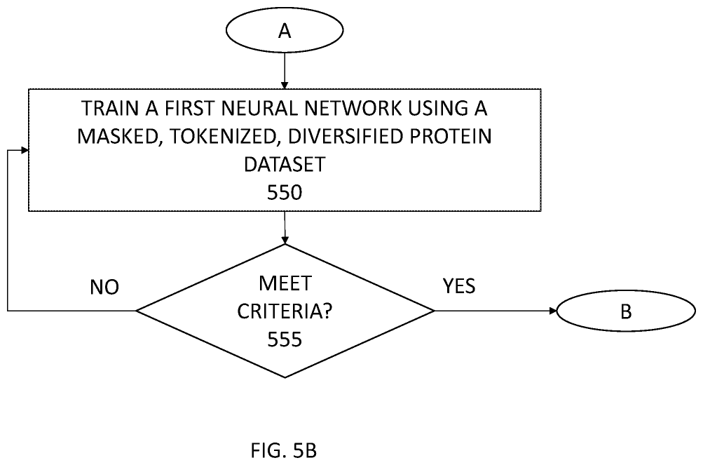

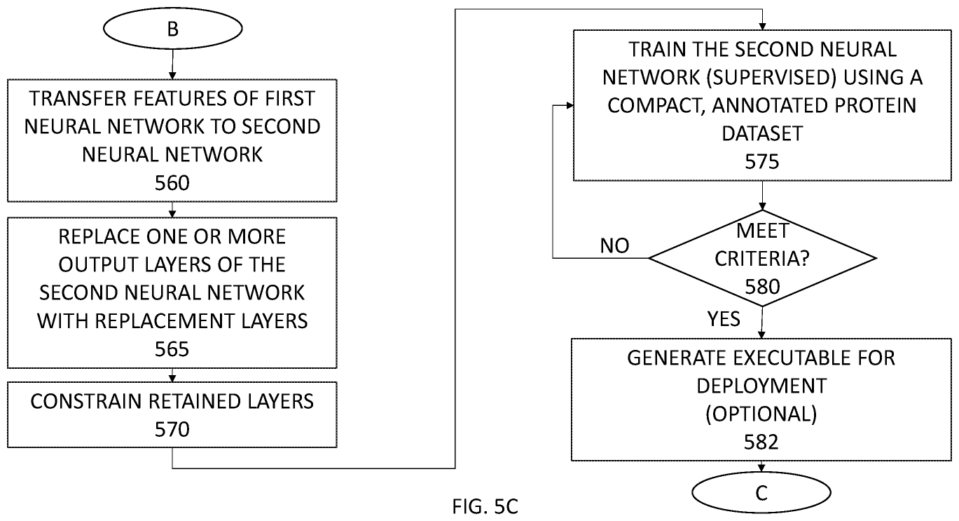

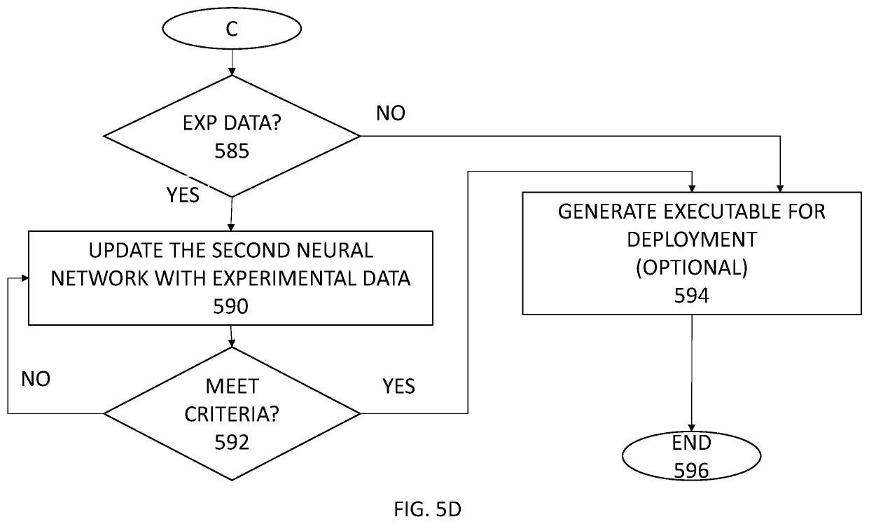

FIGS. 5A-5D show operations associated with training and/or utilizing the protein language NLP system. FIG. 5A is a flow diagram for operations involving generating a training data set for training a protein language NLP system according to embodiments of the present disclosure. At operation 510, a library of protein sequences is ingested. The libraries may include any suitable protein repository including but not limited to public databases such as UniProt (uniprot.org), EMBL (ebi.ac.uk), PFam (pfam.xfam.org), private databases such as internal databases, or any combination thereof. The protein sequences may be provided in any suitable format including FASTA, SAM, etc. At operation 520, the protein sequences undergo tokenization, for example, individual amino acid-level tokenization, n-mer tokenization or sub-word tokenization. At operation 530, the tokenized sequences are subjected to a masking process, for example, in which about 15% of the amino acids are masked (e.g., 5-25%, 10-20%, 12-17%, 15%). At operation 540, the training dataset is generated for input into the first neural network. Label “A” from FIG. 5A continues to Label “A” on FIG. 5B.

Continuing to FIG. 5B, at operation 550, a first neural network (e.g., one or more transformer models with attention (e.g., self-attention)) is trained, e.g., in a self-supervised manner, using a masked, tokenized, protein dataset (e.g., diverse proteins in some aspects; in other aspects, TCRs and epitopes). At operation 555, the system determines whether suitable training criteria, such as AUC-ROC criteria, have been met. If not, training may continue at operation 550. Otherwise, training is terminated, and transfer learning proceeds. Label “B” from FIG. 5B continues to Label “B” on FIG. 5C.

With respect to FIG. 5C, at operation 560, the parameters of the trained first neural network are transferred to a second neural network. In one aspect, a second neural network may be generated, and the features (e.g., configuration, embeddings, layers, variables or hyperparameters) of the first neural network are loaded into the second neural network. Alternatively, in another aspect, operations may proceed with fine-tuning the second neural network (obtained from the first phase), which is trained in a second phase with a compact annotated dataset. (In this case, the trained first neural network or features thereof is stored to allow fine-tuning for other applications.) At operation 565, one or more output layers of the second neural network are replaced with one or more replacement layers that have not been trained. At operation 570, the retained layers, which are the layers retained from training of the first neural network, are constrained to preserve information from the first phase of training. At operation 575, the second neural network is trained using a specific, compact annotated dataset in a supervised manner. The replacement layers are trained to predict the biophysiochemical properties, and the retained layers are subject to model constraints 264, which may be relaxed as training progresses. At operation 580, the system determines whether suitable training criteria have been met (e.g., AUC-ROC, etc.). If not, training may continue at operation 575. If criteria have been met, training is terminated. In some aspects, at operation 582, an executable for deployment on a client system may be generated (optional). Label “C” of FIG. 5C continues to Label “C” on FIG. 5D.

With reference to FIG. 5D, at operation 585, the system determines whether experimental data is available. If no data is available, training operations may cease. At operation 594, an executable for deployment may be generated (optional), and at operation 596, the process ends.

If data is available, additional training of the second neural network may occur with the experimental data at operation 590. At operation 592, the system determines whether suitable training criteria have been met. If not, training may continue at operation 590. Otherwise, training is terminated. At operation 594, an executable for deployment may be generated (optional). The process ends at operation 596. Operations 585-596 may be repeated as additional data becomes available.

Additionally, the first neural network may be updated as new protein sequences become available, and the second neural network retrained accordingly.

FIGS. 5A-5D provide flow diagrams for generating a trained protein language NLP system, according to embodiments of the present disclosure. These diagrams show operations associated with data undergoing a tokenization and masking process from a diverse protein dataset. The first neural network is trained, and features from this first neural network are transferred to a second neural network. The second neural network is fine-tuned by training with a specific annotated dataset in a supervised manner to predict a desired biophysiochemical property.

FIG. 6 shows output probabilities at masked amino acid positions, according to embodiments of the present disclosure, based on the first phase of training the protein language NLP system.

FIG. 7 is an example of transfer learning according to the embodiments provided herein. This example is intended to represent one of many ways that transfer learning may be performed and is not intended to be limiting.

In this example, the first neural network is a transformer model with attention, trained on a diversified, tokenized and masked dataset of protein sequences. Once the first phase of training is complete, the knowledge gained from the first phase of training is transferred to a second neural network using transfer learning.

In some aspects, to achieve transfer of knowledge, the first neural network may be copied to a second neural network or the parameters and configurations of the first neural network may be loaded into a second neural network. The one or more output layers of the second neural network may be truncated and replaced with replacement layers that have not previously been subjected to training (truncated neural network). In some aspects, the replacement layer(s) may comprise a shallow classifier. In this example, a truncated neural network is generated that comprises retained layers from the trained first neural network and replacement layers which are untrained layers. Thus, the first neural network is maintained for subsequent applications, and a second neural network is generated and trained. By restricting or “freezing” the retained neural network layers containing information from the first phase of training, and allowing modification of the replacement layers during the second phase of training, knowledge is transferred and retained from the first phase of training, while allowing customization of the second neural network to a specific application (e.g., prediction of a specific biophysiochemical property) by training with an annotated, compact dataset. Thus, this approach allows retention of features from the first phase of training, while allowing customization during a subsequent phase of training.

Alternatively, the output of the first and second transformer may be transferred to a second neural network in any suitable manner, for example, in the form of information/variables (such as embeddings, categorical variables, hyperparameters, concatenated representations of sequence and categorical feature embeddings, layers or combinations thereof, etc.).

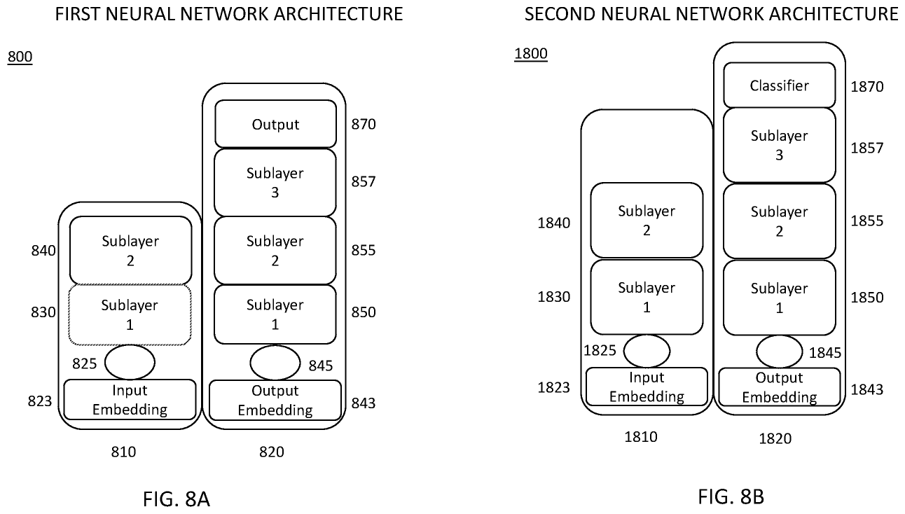

FIGS. 8A and 8B show example architectures of attention-based neural networks. This example is intended to represent one of many neural network architectures compatible with the protein language NLP system and is not intended to be limiting. In this example, both neural networks may comprise a transformer model with attention.

In an aspect, the transformer model 800 has at least one encoder 810 and at least one decoder 820. The encoder may comprise a plurality of encoder layers (Nₓ), and the decoder may comprise a plurality of decoder layers (Nₓ). An encoder layer comprises two sublayers, sublayer 1 830 comprising a multi-head attention layer and a sublayer 2 840 comprising a fully connected feedforward layer. A positional encoder 825 is included between input embedding 823 and sublayer 1 830. A decoder layer comprises three sublayers, sublayer 1 850 comprising a masked multi-head attention layer, a sublayer 2 855 comprising a multi-head attention layer, and sublayer 3 857 comprising a feedforward layer. A positional encoder 845 is included between input embedding 843 and sublayer 1 850. At the output of the decoder, an output layer 870 may be applied to generate output probabilities.

In aspects, the at least one encoder is coupled to the at least one decoder, such that the output of the feedforward layer of the encoder is provided as input to the multi-head attention layer of the decoder. The multi-head attention layer allows the transformer to jointly attend to information from different representation subspaces at different positions, and also directs which parts of the input vector the neural network focuses on to generate the output vector. The feed forward layer is fully connected and may feed forward data after adjustments with biases and weightings. The masked multi-head attention layer prevents the decoder of the neural network from seeing “future” values that should not be seen during training Positional encoding at the input and output allows the transformer to capture ordering information of a sequence of amino acids.

Transformer models with attention have been applied to the English language (see, Vaswani et al., “Attention is All you Need,” (2019) arXiv:1706.03762v5). However, the applicability of such models to non-language analysis is not well-understood.

In FIG. 8B, the transformer model 1800 has at least one encoder 1810 and at least one decoder 1820. The encoder may comprise a plurality of encoder layers (Nₓ), and the decoder may comprise a plurality of decoder layers (Nₓ). In some aspects, an encoder layer 1810 comprises two sublayers, a sublayer 1 1830 comprising a multi-head attention layer and sublayer 2 1840 comprising a fully connected feedforward layer. A positional encoder 1825 is included between input embedding 1823 and sublayer 1 1830. A decoder layer 1820 comprises three sublayers, sublayer 1 1850 comprising a masked multi-head attention layer, sublayer 2 1855 comprising a multi-head attention layer, and sublayer 3 1857 comprising a feedforward layer. A positional encoder 1845 is included between input embedding 1843 and sublayer 1 1850. A classifier 1870 may be trained to predict a biophysiochemical property.

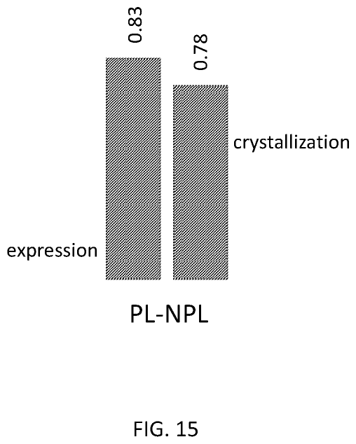

Here, it is surprisingly found that the protein language NLP system has successfully predicted a wide variety of biophysiochemical properties (see, FIGS. 15-21) better than corresponding benchmarks.

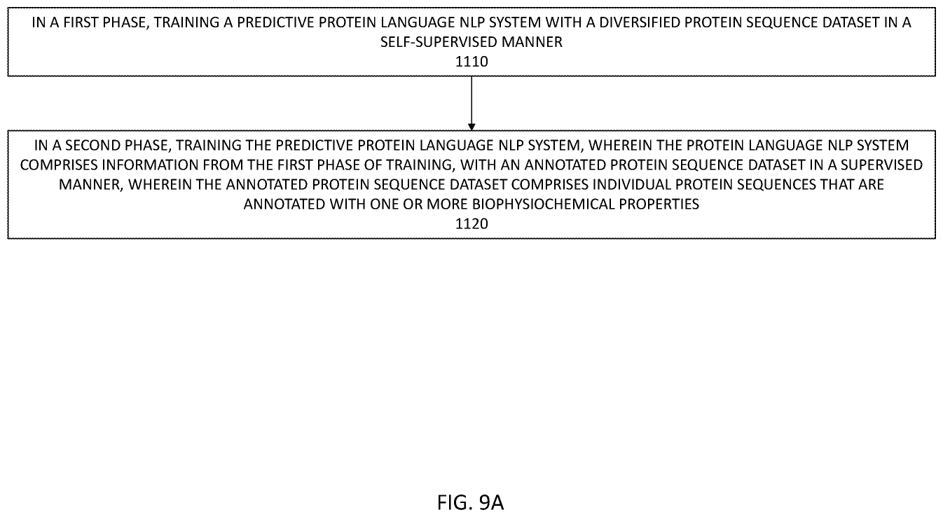

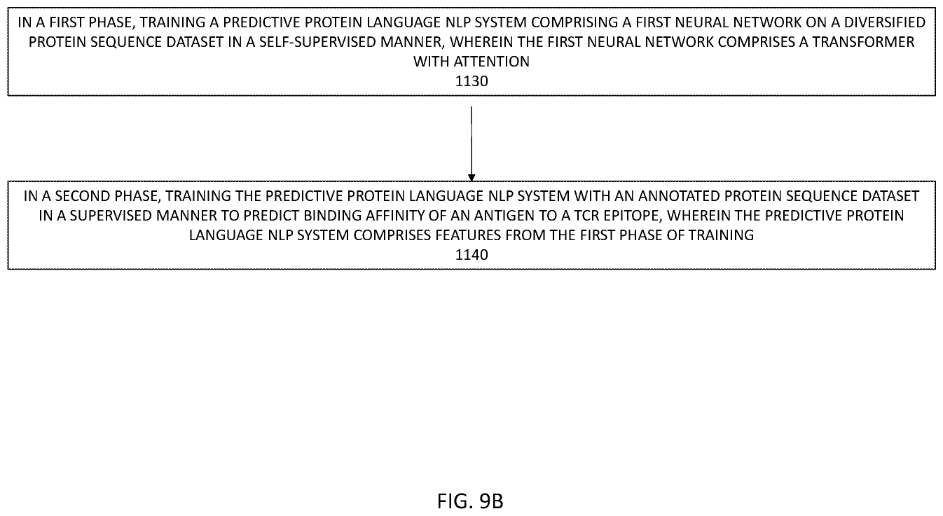

FIGS. 9A-11B show example flowcharts of operations for training and utilizing the protein language NLP system according to the embodiments provided herein.

FIG. 9A is a high-level flowchart of example training operations of the predictive protein language NLP system, in accordance with the embodiments provided herein. At operation 1110, in a first phase, a predictive protein language NLP system is trained with a (e.g., diversified) protein sequence dataset in a self-supervised manner. At operation 1120, in a second phase, the predictive protein language NLP system, wherein the predictive protein language NLP system comprises features from the first phase of training, is trained with an annotated protein sequence dataset in a supervised manner, wherein the annotated protein sequence dataset comprises individual protein sequences that are annotated with one or more biophysiochemical properties.

FIG. 9B is another flowchart of example operations for training a predictive protein language NLP system that predicts a binding affinity of an antigen to a TCR epitope, in accordance with the embodiments provided herein. At operation 1130, in a first phase, a predictive protein language NLP system comprising a first neural network is trained on a protein sequence dataset in a self-supervised manner, wherein the first neural network comprises at least one transformer with attention. At operation 1140, in a second phase, the predictive protein language NLP system is trained with an annotated protein sequence dataset in a supervised manner to predict a binding affinity of an epitope to a TCR, wherein the predictive protein language NLP system comprises features from the first phase of training.

In other aspects, in a first phase and with respect to the first neural network, a first transformer is trained on masked, tokenized TCRs and a second transformer is trained on masked, tokenized epitopes. Additional information (e.g., variables, labels) may be provided to the first neural network. Once trained, the first neural network may generate concatenated representations of sequence and categorical feature embeddings that are transferred to a second neural network. the second neural network may undergo fine-tuning to predict certain biophysical properties (e.g., TCR epitope bindings).

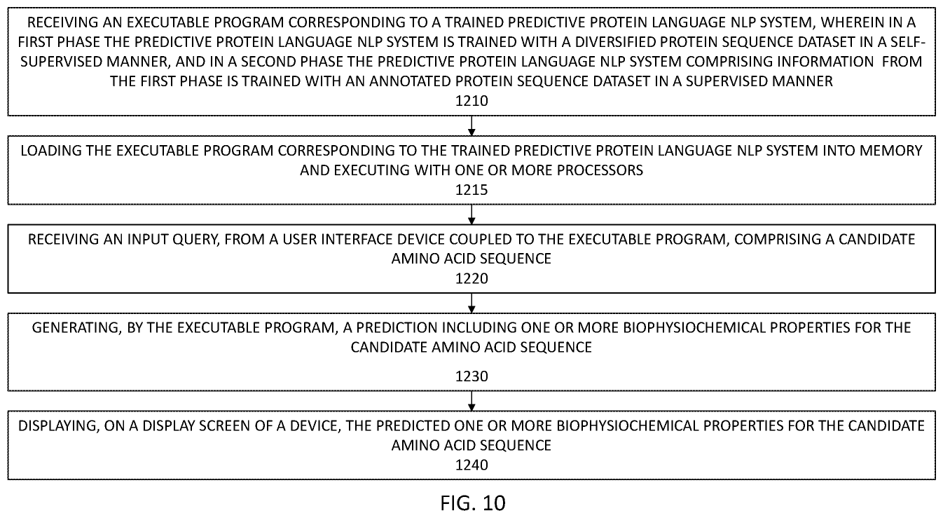

FIG. 10 is a flowchart of example operations for an executable corresponding to a trained predictive protein language NLP system, in accordance with the embodiments provided herein. At operation 1210, an executable program corresponding to a trained predictive protein language NLP system is received, wherein in a first phase the predictive protein language NLP system is trained with a diversified protein sequence dataset in a self-supervised manner, and in a second phase the predictive protein language NLP system comprising information from the first phase is trained with an annotated protein sequence dataset in a supervised manner, and wherein the annotated protein sequence dataset comprises individual protein sequences that are annotated with one or more biophysiochemical properties. At operation 1215, the executable program corresponding to the trained predictive protein language NLP system is loaded into memory and is executed with one or more processors. At operation 1220, an input query is received from a user interface device coupled to the executable program, with the input query comprising a candidate amino acid sequence. At operation 1230, the executable program generates a prediction including one or more biophysiochemical properties for the candidate amino acid sequence. At operation 1240, the predicted one or more biophysiochemical properties for the candidate amino acid sequence is displayed on a display screen of a device.

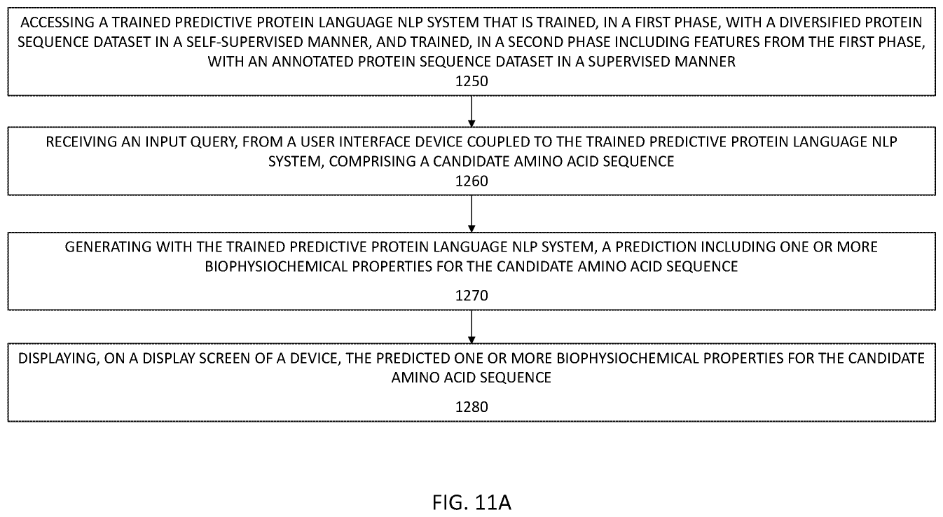

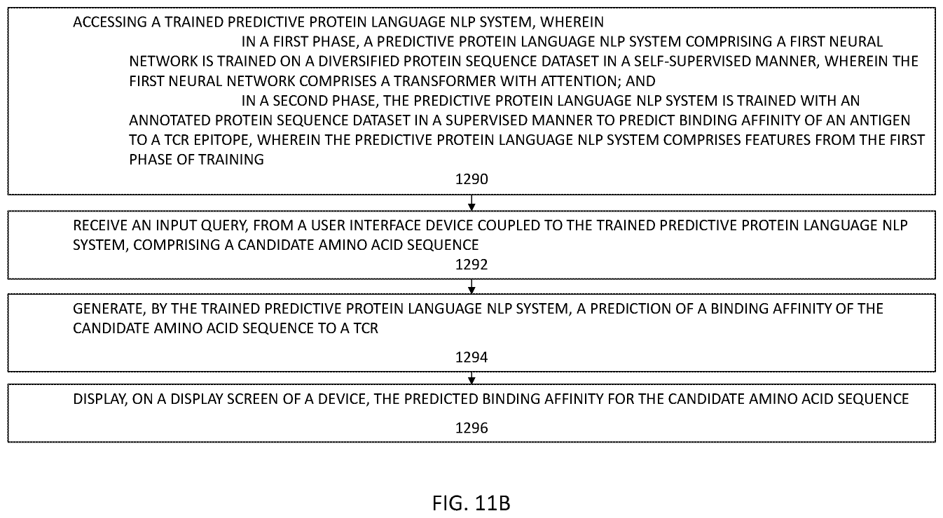

FIG. 11A is a flowchart of example operations for a trained predictive protein language NLP system, in accordance with the embodiments provided herein. At operation 1250, a trained predictive protein language NLP system is provided, that is trained, in a first phase, with a diversified protein sequence dataset in a self-supervised manner, and trained, in a second phase and including features from the first phase, with an annotated protein sequence dataset in a supervised manner. The annotated protein sequence dataset comprises individual protein sequences that are annotated with one or more biophysiochemical properties. At operation 1260, an input query is received, from a user interface device coupled to the trained predictive protein language NLP system, comprising a candidate amino acid sequence. At operation 1270, the trained predictive protein language NLP system generates a prediction including one or more biophysiochemical properties for the candidate amino acid sequence. At operation 1280, the predicted one or more biophysiochemical properties for the candidate amino acid sequence is displayed on a display screen of a device.

FIG. 11B is another flowchart of example operations for accessing a trained predictive protein language NLP system that predicts a binding affinity of an epitope to a TCR, in accordance with the embodiments provided herein. At operation 1290, a trained predictive protein language NLP system is accessed, wherein the trained NLP system is trained in a first phase and a second phase. In the first phase, the predictive protein language NLP system comprises a first neural network that is trained on a diversified protein sequence dataset in a self-supervised manner, wherein the first neural network comprises a transformer with attention. In a second phase, the predictive protein language NLP system is trained with an annotated protein sequence dataset in a supervised manner to predict a binding affinity of an antigen to a TCR epitope, wherein the predictive protein language NLP system comprises features from the first phase of training. At operation 1292, an input query comprising a candidate amino acid sequence is received from a user interface device coupled to the trained predictive protein language NLP system. At operation 1294, the trained predictive protein language NLP system generates a prediction of a binding affinity of a candidate amino acid sequence (e.g., epitope) to a TCR. At operation 1296, a display screen of a device displays the predicted binding affinity for the candidate amino acid sequence.

The output of the protein language NLP system comprises a prediction of a biophysiochemical property or a degree of a predicted biophysiochemical property. For example, in some aspects, the prediction may be expression of a protein, stability of a protein, fluorescence of a protein, or binding between a protein and an antigen (e.g., TCR epitope binding). In still other aspects, the output may comprise a prediction of binding affinity.

In aspects, the protein language NLP system allows a library of therapeutic candidates (e.g., vaccines, antigens, etc.) to be provided as input into a trained protein language NLP system, and for the system to predict which, if any, of the therapeutic candidates have a desired biophysiochemical property. In aspects, the same library of therapeutic candidates may be provided as input to different protein language NLP systems tailored to different applications. The results may be combined to allow therapeutic candidates to be selected based upon a combination of predicted biophysiochemical properties.

In aspects, the library of therapeutic candidates may be hypothetical (not yet synthesized) and the output of the protein language NLP system may select therapeutic candidates predicted to have certain properties. These candidates may be synthesized and experimentally validated.

In other aspects, the output of the protein language NLP system may select experimentally validated therapeutic candidates predicted to have certain properties compatible with downstream manufacturing processes.

In aspects, the protein language NLP system may be applied to the field of biotechnology and adapted to the specific technical implementation of predicting a biophysiochemical property for an amino acid sequence. In aspects, the predicted biophysiochemical property is TCR epitope binding and/or a degree of binding, e.g., with a confidence interval according to an AUC criteria determined by statistical approaches.

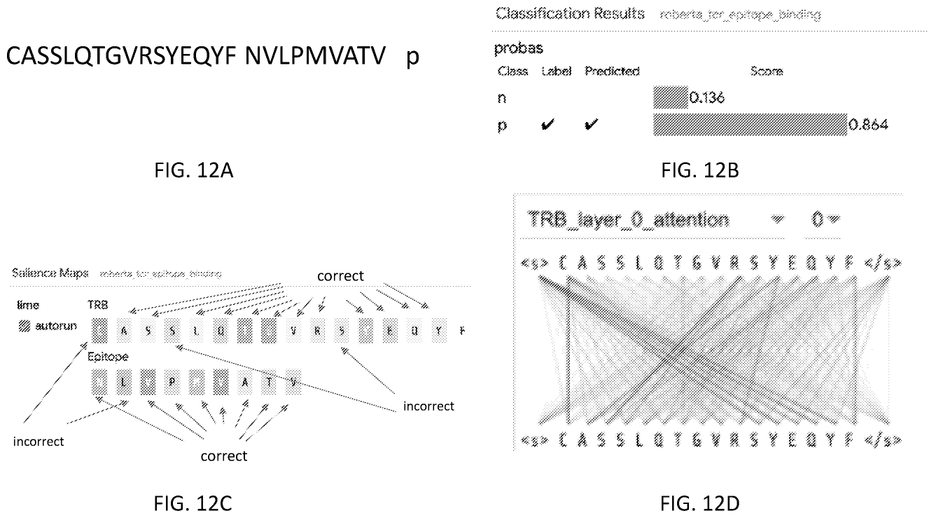

FIGS. 12A-12D show various aspects of user interfaces suitable for displaying and interpreting results generated by the protein language NLP system. In aspects, a training dataset may be used to fine-tune the protein language NLP system. In FIG. 12A, a user may enter novel candidate sequences (e.g., TRB and epitope sequences), previously unknown to the protein language NLP system, and generate binding predictions that appear in FIG. 12B.

FIG. 12B shows a view of classification results for epitopes, for example, whether an epitope binds to a TCR. FIG. 12C shows a view of a salience module showing a map of epitope-TCR binding. In this example, the TRB portion of the TCR is shown along with an epitope predicted to bind to the TRB portion. Individual amino acid residues may be color coded or shaded (grayscale) to illustrate the contribution of each amino acid to binding between the TCR and epitope. For example, a color coding scale/grayscale or other visualization technique may show strength of interaction, representing amino acids from least contributing to highly contributing. In this example, residues contributing to binding are shown as correct, and residues not contributing to binding are shown as incorrect. This feature allows insight into the predictions made by the system, and allows ease of fine-tuning epitopes to improve binding prediction scores. FIG. 12D shows an attention layer or connectivity between individual amino acid residues.

The output of the protein language NLP system may be provided in any suitable manner, including visually on a display, by an audio device, etc.

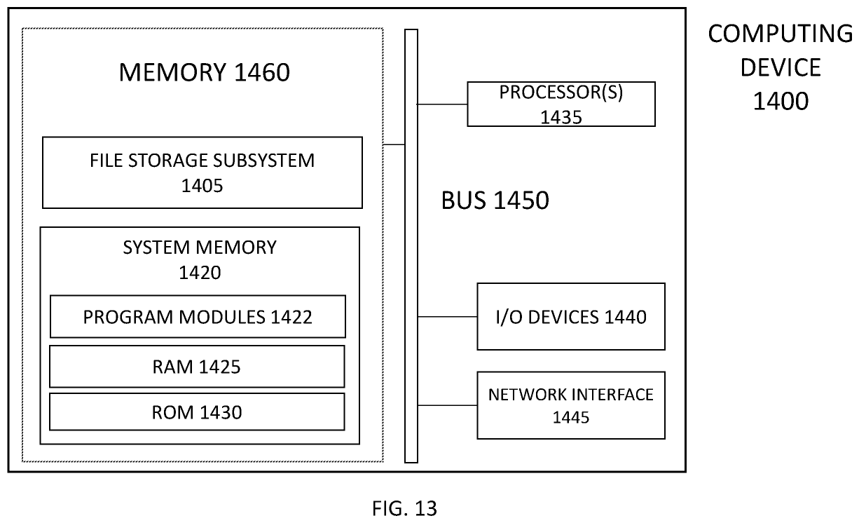

FIG. 13 is an example architecture of a computing device 1400 that may be used to perform one or more aspects of the protein language NLP system described herein. Components of computing device 1400 may include, without limitation, one or more processors (e.g., CPU, GPU and/or TPU) 1435, network interface 1445, I/O devices 1440, memory 1460, and bus subsystem 1450. Each component is described in additional detail below.

It is to be understood that the software (e.g., protein language NLP system of the present embodiments) may be implemented in any desired computer language and may be developed by one of ordinary skill in the computer arts based on the functional descriptions contained in the specification and flow charts illustrated in the drawings. Further, any references herein of software performing various functions generally refer to computer systems or processors performing those functions under software control. The computer systems of the present embodiments may alternatively be implemented by any type of hardware and/or other processing circuitry.

In aspects, the client device may be located in a first geographical location, and the server device housing the protein language NLP system may be located in a second geographical location. In another aspect, the client device and the server device housing the protein language NLP system may be located within a defined geographical boundary. In another aspect, an executable corresponding to the trained protein language NLP system may be generated in a first geographical location, and downloaded and executed by a client device in a second geographical location.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flow charts may be modified in any manner that accomplishes the functions described herein. In addition, the functions in the flow charts or description may be performed in any order that accomplishes a desired operation.

It should be appreciated that all combinations of the foregoing concepts and additional concepts are contemplated as being part of the subject matter disclosed herein. All combinations of claimed subject matter appearing at the end of this disclosure are contemplated as being part of the subject matter disclosed herein. It will be appreciated that the embodiments described above and illustrated in the drawings represent only a few of the many ways of combining the features provided herein.

Computing device 1400 can any suitable computing device including a workstation, server, computing cluster, blade server, server farm, or any other data processing system or computing device. The example description of computer device 1400 depicted in FIG. 13 is intended only for purposes of illustrating some implementations. It is understood that many other configurations of computer system 1400, for example, with more or less components than as depicted in FIG. 13 are possible and fall within the scope of the embodiments provided herein.

It should also be understood that, although not shown, other hardware and/or software components may be used in conjunction with computing device 1400. Examples, include, but are not limited to: redundant processing units, external disk drive arrays, RAID systems, data archival storage systems, etc.

### Memory

Memory 1460 stores programming instructions/logic and data constructs that provide the functionality of some or all of the software modules/programs described herein. For example, memory 1460 may include the programming instructions/logic and data constructs associated with the protein language NLP system to perform aspects of the methods described herein. The programming instructions/logic may be executed by one or more processor(s) 1435 to implement one or more software modules as described herein. In embodiments, computing device 1400 may have multiple processors 1435, and/or multiple cores per processor.

Programming instructions/logic and data constructs may be stored on computer readable storage media. Unless indicated otherwise, a computer readable storage medium is a tangible device that retains and stores program instructions/logic for execution by a processor device (e.g., CPU, GPU, controller, etc.).

Memory 1460 may include system memory 1420 and file storage subsystem 1405, which may include any suitable computer readable storage media. In aspects, system memory 1420 may include RAM 1425 for storage of program instructions/logic and data during program execution and ROM 1430 used for storage of fixed program instructions. The software modules/program modules 1422 of the protein language NLP system contain program instructions/logic that implement the functionality of embodiments provided herein, as well as any other program or operating system instructions, and may be stored in system memory 1420 or in other devices or components accessible by the processor(s) 1435. File storage system 1405 may include, but is not limited to, a hard disk drive, a disk drive with associated removable media, or removable media cartridges, and may provide persistent storage for program instruction/logic files and/or data files.

A non-exhaustive list of examples of computer readable storage media may include any volatile, non-volatile, removable, or non-removable memory, such as: a random access memory (RAM), a dynamic random access memory (DRAM), a static random access memory (SRAM), a cache memory, a read-only memory (ROM), an erasable programmable read-only memory (EPROM or Flash memory), a portable computer diskette, a portable compact disc read-only memory (CD-ROM), a digital versatile disk (DVD), a floppy disk, a memory stick, or magnetic storage, a hard disk, hard disk drives (HDDs), or solid state drives (SSDs).

A computer readable storage medium is not to be construed as transitory signals, such as electrical signals transmitted through a wire, freely propagating electromagnetic waves, light waves propagating along a fiber, or freely propagating radio waves. The computer readable storage medium provided herein is non-transitory.

### I/O Devices

Input/output (I/O) device(s) 1440 may include one or more user interface devices that enable a user to interact with computing device 1400 via input/output (I/O) ports. User interface devices, which include input and output devices, may refer to any visual and/or audio interface or prompt with which a user may use to interact with computing device 1400. In some aspects, user interfaces may be integrated into executable software applications, programmed based on various programming and/or scripting languages, such as C, C #, C++, Perl, Python, Pascal, Visual Basic, etc. Other user interfaces may be in the form of markup language, including HTML, XML, or VXML. The embodiments described herein may employ any number of any type of user interface devices for obtaining or providing information.

Interface input devices may include any number of any types of input or actuation mechanisms (e.g., buttons, icons, fields, boxes, links, etc.) to enter/display information and initiate desired actions via any suitable input devices (e.g., mouse, keyboard, etc.). The interface screens may include any suitable actuators (e.g., links, tabs, etc.) to navigate between the screens in any fashion. User interface input devices may include a keyboard, pointing devices such as a mouse, a trackball, a touchpad, a graphics tablet, a scanner, a touchscreen incorporated into the display, audio input devices such as voice recognition systems, microphones, and/or any other suitable type of input device. In general, use of the term “input device” is intended to include all possible types of devices and ways to input information into computer device 1400 or any other suitable input device for receiving inputs from a user.

User interface output devices may include a display, including a cathode ray tube (CRT) display, a light emitting diode (LED) display, a liquid crystal display (LCD), an organic LED (OLED) display, a plasma display, a projection device, or other suitable device for generating a visual image. User interface output devices may include a printer, a fax machine, a display, or non-visual displays such as audio output devices. In general, use of the term “output device” is intended to include all possible types of devices and ways to output information from computer device 1400 to the user or to another machine or computer system.

### Network Interface

Computing device 1400 can communicate with one or more networks such as a local area network (LAN), a general wide area network (WAN), and/or a public or private network (e.g., the Internet, an Intranet, etc.) via network interface 1445. In some aspects, the network interface 1445 may be a wired communication interface that includes Ethernet, Gigabit Ethernet, or any suitable equivalent. In other embodiments, the network interface 1445 may be a wireless communication interface that includes modulators, demodulators, and antennas for a variety of wireless protocols including, but not limited to, Bluetooth, Wi-Fi, and/or cellular communication protocols for communication over a computer network. Network interface 1445 is accessible via bus 1450. As depicted, network interface 1445 communicates with the other components of computing device 1400, including processor 1435 and memory 1460, via bus 1450. The network interface allows the computing device 1400 to send and receive data through any suitable network.

### Bus

Bus subsystem 1450 couples the various computing device components together, allowing communication between various components and subsystems of memory 1460, processors 1435, network interface 1445, and I/O devices 1440.

Bus 1450 is shown schematically as a single bus, however, any combination of buses may be used with present embodiments. Bus 1450 represents one or more of any suitable type of bus structure, including a memory bus, a peripheral bus, an accelerated graphics port, or a local bus using any of a variety of bus architectures. By way of example, and without limitation, bus architectures may include Enhanced ISA (EISA) bus, Industry Standard Architecture (ISA) bus, Micro Channel Architecture (MCA) bus, Peripheral Component Interconnects (PCI) bus, and Video Electronics Standards Association (VESA) local bus.

### Program Instructions

The computer program product may include a computer readable storage medium (or media) having computer readable program instructions thereon for causing a processor to carry out aspects of the present invention.

Program modules 1422 may be stored in system memory 1420 by way of example, and not limitation, as well as an operating system, one or more application programs, other program modules, and program data. Program modules 1422 generally carry out the functions and/or methodologies of embodiments as described herein.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other devices implement the functions/acts specified in the flowchart and/or block diagram block or blocks.

Computer readable program instructions for carrying out operations of embodiments of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language, programming languages for machine learning or artificial intelligence such as C++, Python, Java, C, C #, Scala, CUDA or similar programming languages.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), ASICS, or programmable logic arrays (PLA) may execute the computer readable program instructions.

These computer readable program instructions may be provided to a processor of a computer, or other programmable data processing apparatus to produce a machine, such that the instructions, which execute via the processor of the computer or other programmable data processing apparatus, implement the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

Hardware and/or Software

Some of the functional components described in this specification have been labeled as systems or units in order to more particularly emphasize their implementation independence. A system or unit may be implemented as a hardware circuit (e.g., custom VLSI circuits or gate arrays, off-the-shelf semiconductors such as logic chips, transistors, or other discrete components, etc.) or in programmable hardware devices (e.g., field programmable gate arrays, programmable array logic, programmable logic devices, etc.). Alternatively, a system or unit may also be implemented in software for execution by various types of processors. For example, a system, unit or component of executable code may comprise one or more physical or logical blocks of computer instructions, which may be organized as an object, procedure, or function. The executables of an identified system or unit need not be physically located together, but may comprise disparate instructions stored in different locations which, when joined logically together, comprise the system or unit and achieve the stated purpose for the system, unit or component.

In general, a hardware element (e.g., CPU, GPU, RAM, ROM, etc.) may refer to any hardware structures arranged to perform certain operations. In one embodiment, for example, the hardware elements may include any analog or digital electrical or electronic elements fabricated on a substrate. The fabrication may be performed using silicon-based integrated circuit (IC) techniques, such as complementary metal oxide semiconductor (CMOS), bipolar, and bipolar CMOS (BiCMOS) techniques, for example. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor devices, chips, microchips, chip sets, and so forth. However, the embodiments are not limited in this context.

Also noted above, some embodiments may be embodied in software. The software may be referenced as a software module or element. In general, a software element may refer to any software structures arranged to perform certain operations. In one embodiment, for example, the software elements may include program instructions and/or data adapted for execution by a hardware element, such as a processor. Program instructions may include an organized list of commands comprising words, values, or symbols arranged in a predetermined syntax that, when executed, may cause a processor to perform a corresponding set of operations.

A system or unit of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices and disparate memory devices.

Furthermore, systems/units may also be implemented as a combination of software and one or more hardware devices.

### General

The terminology used herein is for the purpose of describing particular embodiments only and is not intended to be limiting of the embodiments provided herein. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

Reference throughout this specification to “one embodiment,” “an embodiment,” “some embodiments”, or similar language means that a particular feature, structure, or characteristic described in connection with the embodiment is included in at least one embodiment provided herein and may refer to the same or different embodiments.

While the disclosure outlines exemplary embodiments, it will be appreciated that variations and modifications will occur to those skilled in the art. For example, although the illustrative embodiments are described herein as a series of acts or events, it will be appreciated that the present invention is not limited by the illustrated ordering of such acts or events unless specifically stated. Some acts may occur in different orders and/or concurrently with other acts or events apart from those illustrated and/or described herein, in accordance with the embodiments. For example, the functions noted in the blocks may occur out of the order noted in the Figures. For example, two blocks shown in succession may, in fact, be executed substantially concurrently, or the blocks may sometimes be executed in the reverse order, depending upon the functionality involved.

Aspects of the present techniques are described herein with reference to flowchart illustrations and/or block diagrams of methods, apparatus (systems), and computer program products. The flowchart and block diagrams in the Figures illustrate the architecture, functionality, and operation of possible implementations of systems, methods, and computer program products according to various embodiments of the present techniques. In this regard, each block in the flowchart or block diagrams may represent a module, segment, or portion of instructions, which comprises one or more executable instructions for implementing the specified logical function(s). It will be understood that each block of the flowchart illustrations and/or block diagrams, and combinations of blocks in the flowchart illustrations and/or block diagrams, can be implemented by computer readable program instructions. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Moreover, in particular regard to the various functions performed by the above described components (assemblies, devices, circuits, etc.), the terms used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments.

The embodiment was chosen and described in order to best explain the principles of the invention and the practical application, and to enable others of ordinary skill in the art to understand the invention for various embodiments with various modifications as are suited to the particular use contemplated.

A variety of other means and/or structures for performing the function and/or obtaining the results and/or one or more of the advantages described herein may be utilized, and each of such variations and/or modifications is deemed to be within the scope of the implementations described herein. More generally, all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific implementations described herein. It is, therefore, to be understood that the foregoing implementations are presented by way of example only and that, within the scope of the appended claims and equivalents thereto, implementations may be practiced otherwise than as specifically described and claimed. The description of the various embodiments herein has been presented for purposes of illustration and description, but is not intended to be exhaustive or limited to the invention in the form disclosed. It will be apparent to those skilled in the art that various modifications and variations can be made to the embodiments provided herein without departing from the spirit and scope of the invention.

### EXAMPLES

### Advantages

For machine learning approaches to be successful, systems are often trained with large volumes of annotated data, which can be time-consuming to generate. Additionally, traditional one-shot approaches may adjust parameters and iteratively retrain systems with the entire dataset until meeting specified criteria.

In contrast, present approaches utilize a different approach relying on the primary structure of proteins. By training a first neural network on a diverse set of protein sequences, the neural network learns rules associated with the ordering of amino acids. This information is transferred to another neural network, which is fine-tuned with a compact, annotated dataset to predict a given biophysiochemical property.

Present approaches provide a variety of technical improvements to the field of machine learning and advance the application of machine learning to biological systems. For example, present approaches accelerate the development and customization of machine learning systems to particular applications. By generally training a first neural network, the machine learning system may be fine-tuned in a second training phase to particular applications in a shorter period of time as compared to one-shot machine learning approaches. This approach allows rapid development of the protein language NLP system to a wide variety of biological applications, including predictions of TCR epitope binding/binding affinity. Subsequent rounds of fine-tuning may also be performed rapidly as additional experimental data becomes available. Thus, present techniques provide a robust and rapid approach to developing and applying machine learning systems to a variety of biological applications.

Further, present approaches greatly reduce the amount of annotated data needed to train a machine learning application for a particular application. For machine learning approaches to be successful, a sufficient amount of data is needed to train the system to meet a specified performance criteria. Training with a small annotated dataset alone may not be sufficient to meet such performance criteria, while generating and annotating large datasets may be time-consuming and costly. In the present case, the protein language NLP system uses primary amino acid sequences obtained from public databases to learn rules of protein sequences, which allows the system to be trained with a smaller compact annotated dataset in the second phase of training. It is surprising that information learned purely based on the amino acid sequence order of proteins in a first phase of training is able to reduce the amount of annotated data that would otherwise be needed to train a machine learning system to predict a biophysiochemical property such as TCR-epitope binding/binding affinity.

Further, and as shown by the following examples, the present approach meets or exceeds current benchmarks by other machine learning approaches for predicting various biochemical properties. Namely, the approach provided herein has provided results that exceed Net-TCR and ERGO benchmarks. In aspects, present approaches that utilize transformer models with attention have demonstrated improved performance over other architecture types including convolutional neural nets (CNNs), shallow neural nets (e.g., Word2Vec), long term short memory (LTSM) networks (e.g., ULMFit), and discriminators (e.g., Electra).

In aspects, a programming environment compatible with NLP techniques may be utilized, and may comprise or be integrated with one or more libraries of various types of neural network models. Database downloads of amino acid sequences may be obtained, and imported into the programming environment as provided herein.

The protein language NLP system may be first trained on a diversified protein sequence repository to learn the language of proteins. Training on a diversified protein dataset ensures that the protein language software is trained on proteins in general, including proteins in different categories with different functions. In some aspects, the diversified protein dataset includes tens of thousands of entries or more. This generalized training accelerates subsequent training to fine-tune the protein language software to a particular category. Once generally trained, the protein language software may be fine-tuned, for example, using a smaller, specific annotated dataset relative to the diversified dataset.

### EXAMPLES

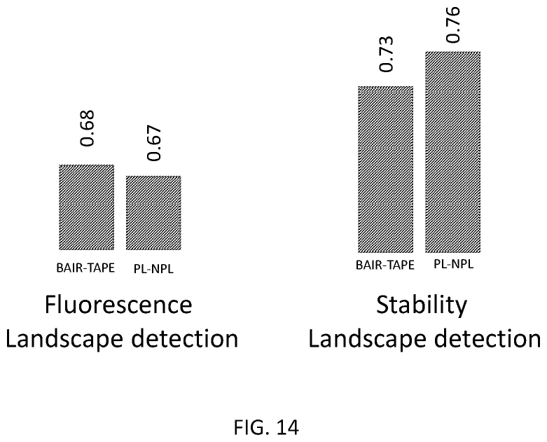

The protein language NLP system may be generally trained and fine-tuned for a variety of applications, including: (1) prediction of protein solubility and stability; (2) predicting protein expression in an expression system, such as prediction of protein expression/yield in organisms such as E. coli; (3) predicting T-cell receptor-epitope binding specificity and prediction of TCR-epitope binding affinity (better than corresponding benchmarks) for human MHC class I restricted epitopes by training with publicly available databases (e.g., VDJDB and EIDB); (4) protein crystallization; (5) immunogenic potential; (6) bacterial antigen discovery; (6) protein fluorescence landscape; (7) protein secondary structure (TAPE); (8) protein remote homology; and (9) vaccine design and synthesis (vaccine characterization, vaccine synthesis, structure function relationship), etc. Present techniques are suitable for a wide range of applications. Representative examples are provided below. The examples provided herein may apply to any biophysiochemical property influenced by protein sequence and are not intended to be limiting. FIGS. 14-20 show various aspects of the protein language NLP system including predictions, architectures, and comparisons to various benchmarks.

### Prediction of Fluorescence and Stability

FIG. 14 is a bar graph showing performance aspects of predicting fluorescence and stability characteristics by the protein language NLP system as compared to a benchmark in accordance with certain aspects of the present disclosure. For example, the protein language NLP system (PL-NLP) achieved a performance that is comparable to benchmarked standard (BAIR-TAPE) for fluorescence landscape detection, and exceeded the benchmarked standard (BAIR-TAPE) for stability prediction.

In some aspects, perplexity (random families from BAIR-TAPE's PFam dataset split) showed at least a 38.4% improvement as compared to benchmarked standards.

### Prediction of Protein Expression and Crystallization

FIG. 15 is a bar graph showing performance aspects of predicting protein expression (left) and protein crystallization (right) by the PL-NLP system in accordance with certain aspects of the present disclosure.

### Prediction of Protein Binding

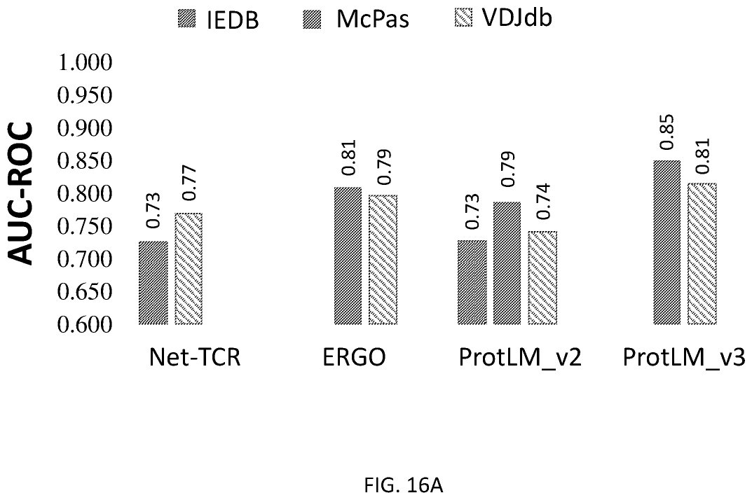

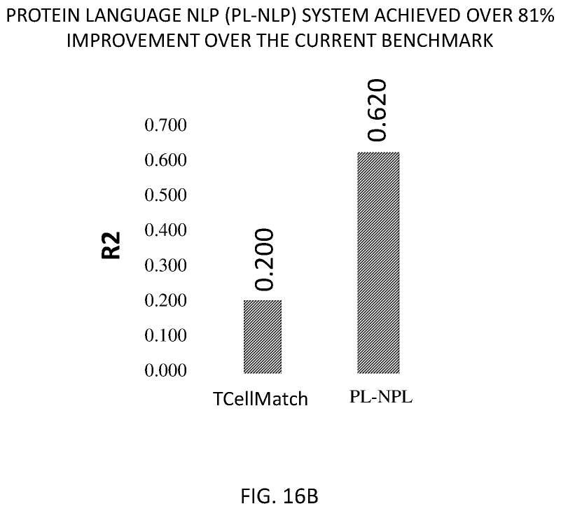

FIG. 16A is a bar graph showing improvements in predicting binding by the protein language NLP system as compared to a benchmark in accordance with certain aspects of the present disclosure. In this figure, the PL-NLP system achieved state-of-the-art performance over benchmarks (e.g., ERGO, Net-TCR) with regard to predicting binding between a TCR sequence and an epitope (see, https://www.frontiersin.org/articles/10.3389/fimmu.2020.01803/full). Different datasets were tested, including IEDB, McPas, and VDJdb covering a diverse set of epitopes. When comparing various datasets (e.g., IEDB, McPas, or VDJdb), PL-NPL* (an earlier version) demonstrated comparable performance to Net-TCR on the IEDB dataset and PL-NLP exceeded benchmarks with regard to ERGO on the McPas dataset and the VDJdb dataset.

FIG. 16B is another bar graph showing prediction of protein binding by the protein language NLP system as compared to a benchmark in accordance with certain aspects of the present disclosure. In this figure, the protein language NLP system demonstrated more than an 81% improvement over the TCellMatch benchmark with regard to predicting binding affinity between a TCR sequence and an epitope, with no additional covariate. PL-NLP has been shown to have an improved predictive ability of predicting binding of a TCR sequence to an epitope, showing a performance improvement of 3× the current benchmark TCellMatch.

### Example Architecture

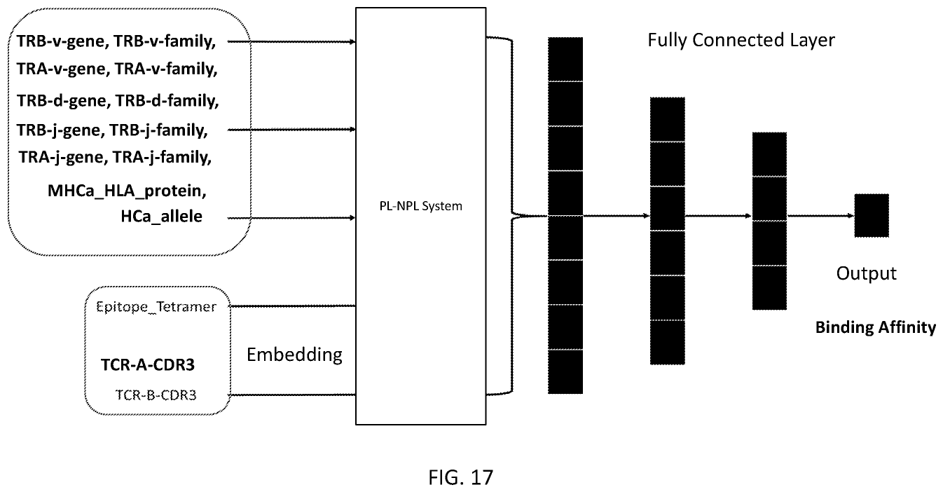

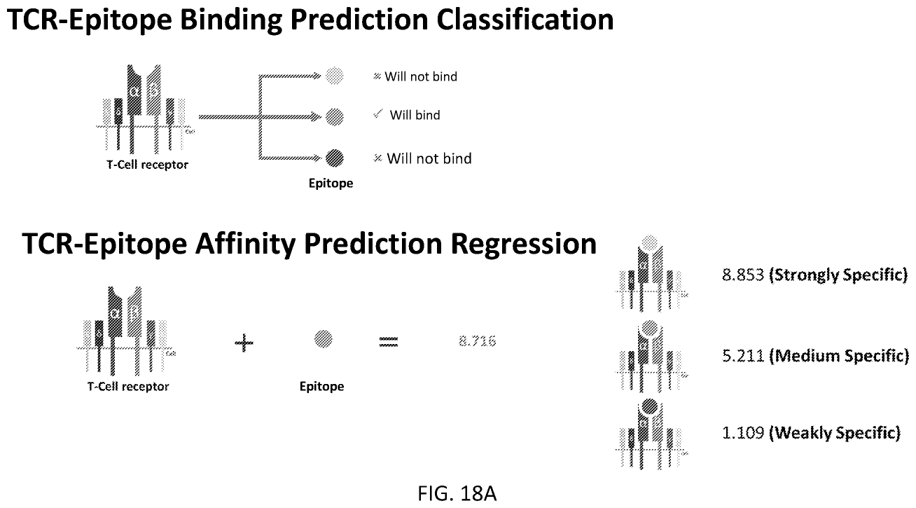

FIG. 17 is an example high-level architecture of the protein language NLP system used for predicting TCR-epitope binding. In this example, various inputs (e.g., TRA-v-gene, TRA-v-family, TRB-v-gene, TRB-v-family, TRB-d-gene, TRB-d-family, TRA-j-gene, TRA-j-family, TRB-j-gene, TRB-j-family, TRA-CDR3 (also referred to as TCR-A-CDR3), TRB-CDR3 (also referred to as TCR-B-CDR3), MHCa_HLA_protein and HCa_allelle, and epitope tetramer) are provided to fully connected layers of a neural network. In aspects, the following are treated as categorical variables: TRA-v-gene, TRA-v-family, TRB-v-gene, TRB-v-family, TRB-d-gene, TRB-d-family, TRA-j-gene, TRA-j-family, TRB-j-gene, TRB-j-family, MHCa_HLA_protein, and HCa_allele. In aspects, the following are treated as embedded variables (by tokenizing on an individual amino acid level): epitope, TCR-A-CDR3, and TCR-B-CDR3. The output is a predicted binding affinity. With present approaches, inputs such as TRA related sequences may easily be included. Predictions may be validated in the wet lab.

### Example Implementation

Datasets were downloaded from publicly available resources (e.g., EMBL, SwissProt, Uniref, etc.). These datasets were tokenized and masked according to the techniques provided herein (e.g., individual amino acid tokenization with about 15% masking of individual amino acids of a protein sequence). This data was utilized to a train a protein language NLP system.

In aspects, a library of transformer models compatible with Python/PyTorch was obtained. In other aspects, transformer models may be developed.

In aspects, a first neural network, such as a robustly optimized bidirectional encoder representation with transformer (RoBERTa) model was trained in a machine learning environment/platform such as Python/PyTorch. In aspects, a four GPU computing system with at least 500 GB RAM was utilized to train the neural network.

During the first phase of training, a RoBERTa model pretraining on UniRef50 (˜37.5M sequences) was used. The following configuration/setup was applied.

Tokenisation: vocabulary size of 32 (character-level IUPAC amino acid codes+special tokens) Architecture (˜38.6M parameters): RoBERTa transformer with: Number of layers: 12 layers; Hidden size: 512; Intermediate size: 2048; Attention heads: 8; Attention dropout: 0.1; Hidden activation: GELU; Hidden dropout: 0.1; Max sequence length: 512.

Self-supervised training (distributed across 8 GPUs with mixed-precision fp16 enabled): Effective batch size: 512 (64 times 8 GPUs); Optimizer: AdamW; Adam epsilon: 1e-6; Adam (beta_1, beta_2): (0.9, 0.99); Gradient clip: 1.0; Gradient accumulation steps: 2; Weight decay: 1e-2; Learning rate: 2e-4; Learning rate scheduler: linear decay with warmup of 1000 steps; Max training steps: 250k (˜7 epochs).

The system may be trained in a second phase of training to be customized to TCR epitope binding (classification) or TCR epitope binding affinity (regression). Data sets including VDJdb, IEDB, McPAS-TCR, and PIRD may be obtained.

Input parameters (various combinations) included:
TCR-B-CDR3 (CDR3b), epitope; or
TCR-B-CDR3 (CDR3b), epitope, MHCa_HLA_protein and HCa_allelle; or
TCR-B-CDR3 (CDR3b), epitope, MHCa_HLA_protein and HCa_allelle, TRB-j-gene, TRB-j-family, TRB-v-gene, TRB-v-family; or
TCR-A-CDR3 (CDR3a), TCR-B-CDR3 (CDR3b), epitope; or
TCR-A-CDR3 (CDR3a), TCR-B-CDR3 (CDR3b), epitope, MHCa_HLA_protein and HCa_allelle.

In aspects, the input parameters included TCR-A-CDR3 (CDR3a), TCR-B-CDR3 (CDR3b), epitope, MHCa_HLA_protein and HCa_allelle, TRB-v-gene, TRB-v-family, TRB-j-gene, TRB-j-family, TRA-v-gene, TRA-v-family, TRA-j-gene, TRA-j-family, TRB-d-gene, TRB-d-family.

In aspects, for classification (prediction of binding), a RoBERTa based model for TCR-epitope binding classification was used. The following setup/configuration was applied:

Tokenisation: vocabulary size of 32 (character-level IUPAC amino acid codes+special tokens).

Architecture: RoBERTa transformer as sequence processing unit (see above) with a final multi-layer perceptron (MLP) as classification head on top of concatenated representations of sequence and categorical feature embeddings.

Supervised training Effective batch size: 128; Optimizer: AdamW; Adam epsilon: 1e-8; Adam (beta_1, beta_2): (0.9, 0.99); Gradient clip: 1.0; Weight decay: 1e-4; Learning rate: 8e-5; Learning rate scheduler: linear decay with warmup of 600 steps; Number of epochs: 12.

In aspects, for regression analysis (a degree of binding), a RoBERTa based model for TCR-epitope regression was used. The following setup/configuration was applied.

Tokenisation: vocabulary size of 32 (character-level IUPAC amino acid codes+special tokens).

Architecture: RoBERTa transformer as sequence processing unit (see above) with a final multi-layer perceptron (MLP) as regression head on top of concatenated sequence and categorical embeddings.

Supervised training (hyperparameters): Effective batch size: 64; Optimizer: AdamW; Adam epsilon: 1e-8; Adam (beta_1, beta_2): (0.9, 0.99); Gradient clip: 1.0; Weight decay: 1e-2; Learning rate: 5e-5; Learning rate scheduler: linear decay; Number of epochs: 4.

Training at any phase proceeded until meeting a specified AUC criteria, for example, greater than 0.65; 0.70; 0.75; 0.80; 0.85; 0.90; 0.95; etc.

Present techniques may also be used for predicting HLA-peptide binding. In other aspects, present techniques may be applied to predict binding of pathogen proteins or peptides to human proteins (e.g., TCRs).

### TCR Binding Affinity

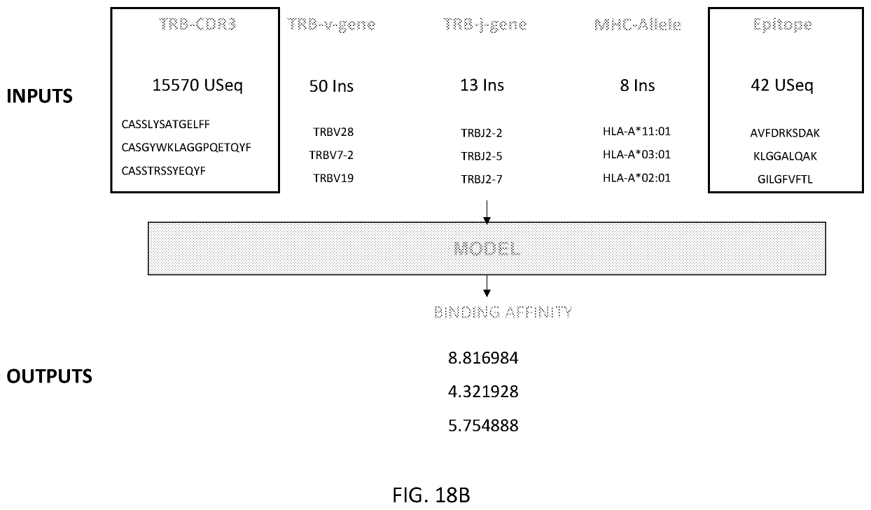

FIG. 18A shows results of TCR-binding prediction classification in accordance with certain aspects of the present disclosure. The top portion of the figure shows a prediction of binding or not binding, while the bottom portion of the figure shows a degree of predicted binding affinity (e.g., strongly specific, medium specific, weakly specific) for a given epitope. The protein language NLP system may classify epitopes into categories such as strongly specific, medium specific, or weakly specific binding affinity based on different threshold cutoff values or ranges. In this example, a TCR-epitope binding affinity of 8.716 was predicted for a given TCR-epitope combination, and example values of binding affinity (8.853, 5.211, 1.109) using regression approaches are provided which fall within respective categories of binding affinity (strongly specific, medium specific, weakly specific).

FIG. 18B shows results of TCR-binding affinity predictions in accordance with certain aspects of the present disclosure. A list of epitopes was provided to the protein language NLP system and binding affinities were predicted for each epitope. In this example, various training sequences (TRB-CDR3, TRB-v-gene, TRB-j-gene, MHC alleles) along with candidate epitopes were provided to the system. The trained system provided as output, predicted binding affinities for the candidate epitopes.

The protein language NLP system, comprising a (TCR-Epitope) classification module, predicted the cognate epitopes of a given TCR from an exhaustive list of published epitopes based on human MHC class I restricted epitopes from publicly available databases (e.g., VDJDB and EIDB). Once classified, the protein sequence software further predicted the binding affinity of a given pair of TCR-epitope sequences (e.g., based on TCR-Epitope Regression techniques).

### Comparison to Other ML Techniques

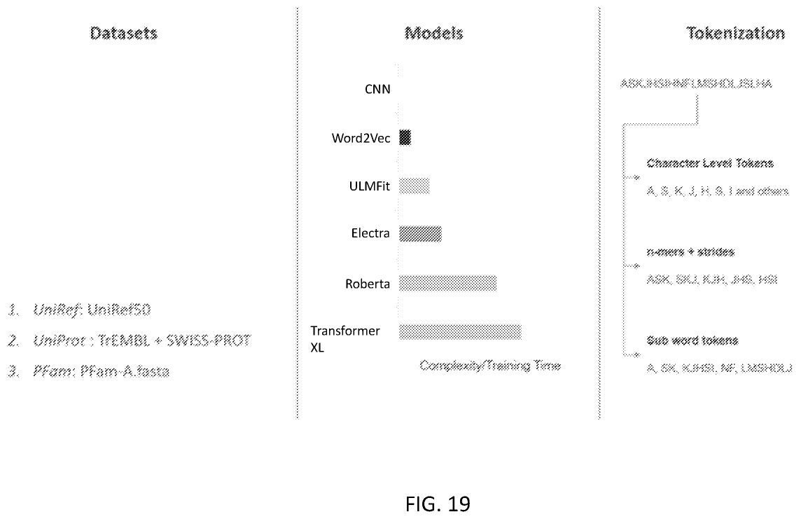

FIG. 19 shows example architectures for the embodiments provided herein. In this example, various datasets are provided to train the protein language NLP system. Any suitable neural network may be used with present techniques, including but not limited to: convolutional neural networks (CNN), Shallow Neural Networks (Word2Vec), LSTMs (e.g., ULMFit), Generative Neural Networks (e.g., Electra), Transformers (e.g., RoBERTa, Transformer-XL), perceptrons, etc. In aspects, transformer models have provided optimal results. In some aspects, the robustly optimized bidirectional encoder representations from transformers model architecture, a transformer model, outperformed other tested approaches. Various tokenization schemes are shown.

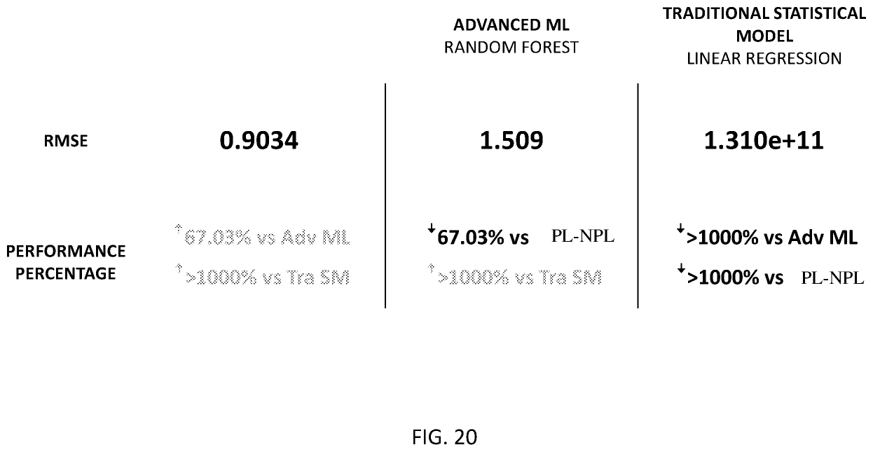

FIG. 20 shows results of a comparison of the protein language NLP system to other machine learning techniques, in accordance with certain aspects of the present disclosure. The protein language NLP system (PL-NLP) outperformed both advanced machine learning techniques (random forest algorithms) and traditional statistical approaches (including linear regression) as evidenced by a lower root mean squared error (RMSE). In particular, the RMSE was 67% reduced for the protein language NLP system as compared to the advanced machine learning model. The RMSE was over 1000% reduced for the protein language NLP system as compared to the statistical regression model. The binding affinity (log read count) of TCR-Epitope binding was predicted with a corresponding Root Mean Square Error (RMSE) of 0.9034, as compared to an RMSE of 1.509 (Random Forest) and 1.3e\hat{ }11 (Linear Regression).

1. A computer-implemented method for training a predictive protein language natural language processing (NLP) system to predict biophysiochemical properties of an amino acid sequence comprising:
in a first phase, training the predictive protein language NLP system comprising a first neural network on a diversified protein sequence dataset in a self-supervised manner, wherein the first neural network comprises one or more transformers with attention; and
in a second phase, training the predictive protein language NLP system in a supervised manner to predict a biophysiochemical property, wherein the predictive protein language NLP system comprises features from the first phase of training.

2. The computer-implemented method of claim 1, wherein the first neural network comprises a first transformer with attention and a second transformer with attention, wherein the first transformer is trained on a first tokenized masked dataset and the second transformer is trained on a second dataset.

3. The computer-implemented method of claim 1, further comprising generating concatenated sequence and categorical embeddings from the first phase of training and providing the concatenated sequence and categorical embeddings to the second neural network for the second phase of training.

4. The computer-implemented method of claim 2, wherein the first transformer or the second transformer comprises a robustly optimized bidirectional encoder representations from transformers model.

5. The computer-implemented method of claim 1, wherein the biophysiochemical property is binding affinity of a TCR to an epitope.

6. The computer-implemented method of claim 1, further comprising:
training, in the first phase, the predictive protein language NLP system using a protein sequence dataset that has undergone individual amino acid-level tokenization, n-mer tokenization, or sub-word tokenization of respective protein sequences.

7. The computer-implemented method of claim 1, further comprising:
training, in the first phase, the predictive protein language NLP system using a diversified protein sequence dataset, wherein about 10-20% of individual amino acids in the diversified protein sequence dataset are masked.

8. A computer-implemented method for predicting biophysiochemical properties of an amino acid sequence using natural language processing (NLP) comprising:
providing a trained predictive protein language NLP system, wherein the trained predictive protein language NLP system is generated by:
in a first phase, training a predictive protein language NLP system comprising a first neural network on one or more protein sequence datasets in a self-supervised manner, wherein the first neural network comprises one or more transformers with attention; and
in a second phase, training the predictive protein language NLP system with a protein sequence dataset in a supervised manner to predict a biophysiochemical property, wherein the predictive protein language NLP system comprises features from the first phase of training;
receiving an input query, from a user interface device coupled to the trained predictive protein language NLP system, comprising a candidate amino acid sequence;
generating, by the trained predictive protein language NLP system, a prediction including one or more biophysiochemical properties for the candidate amino acid sequence; and
displaying, on a display screen of a device, the predicted one or more biophysiochemical properties for the candidate amino acid sequence.

9. (canceled)

10. (canceled)

11. (canceled)

12. The computer-implemented method of claim 8, wherein the biophysiochemical property is binding affinity of a TCR to an epitope.

13. (canceled)

14. (canceled)

15. The computer-implemented method of claim 8, wherein the trained predictive protein language NLP system comprises a salience module, further comprising:
generating, for display on the display screen, information from the salience module that indicates a contribution of respective amino acids to the prediction of the binding affinity.

16. The computer-implemented method of claim 8, wherein the trained predictive protein language NLP system is compiled into an executable file.

17. The computer-implemented method of claim 8, further comprising:
receiving a plurality of candidate amino acid sequences;
analyzing the candidate amino acid sequences; and
predicting whether a candidate epitope binds to a TCR.

18. A system or apparatus to predict biophysiochemical properties of an amino acid sequence comprising one or more processors for executing instructions to:
provide a trained predictive protein language NLP system, wherein:
in a first phase, the trained predictive protein language NLP system comprising a first neural network is trained on a protein sequence dataset in a self-supervised manner, wherein the first neural network comprises one or more transformers with attention; and
in a second phase, the predictive protein language NLP system is trained in a supervised manner to predict a biophysiochemical property, wherein the predictive protein language NLP system comprises features from the first phase of training;
receive an input query, from a user interface device coupled to the trained predictive protein language NLP system, comprising a candidate amino acid sequence;
generate, by the trained predictive protein language NLP system, a prediction including one or more biophysiochemical properties for the candidate amino acid sequence; and
display, on a display screen of a device, the predicted one or more biophysiochemical properties for the candidate amino acid sequence.

19. The system or apparatus of claim 18, wherein the first neural network comprises a first transformer with attention and a second transformer with attention, wherein the first transformer is trained on a first tokenized masked dataset and the second transformer is trained on a second dataset.

20. (canceled)

21. (canceled)

22. The system or apparatus of claim 18, wherein the biophysiochemical property is binding affinity of a TCR to an epitope.

23. (canceled)

24. (canceled)

25. A computer program product for predicting biophysiochemical properties of an amino acid sequence, the computer program product comprising a computer readable storage medium having instructions corresponding to a predictive protein language NLP system embodied therewith, the instructions executable by one or more processors to cause the processors to:
provide a trained predictive protein language NLP system, wherein:
in a first phase, a predictive protein language NLP system comprising a first neural network is trained on a diversified protein sequence dataset in a self-supervised manner, wherein the first neural network comprises one or more transformers with attention; and
in a second phase, the predictive protein language NLP system is trained with an annotated protein sequence dataset in a supervised manner to predict a biophysiochemical property, wherein the predictive protein language NLP system comprises features from the first phase of training;
receive an input query, from a user interface device coupled to the trained predictive protein language NLP system, comprising a candidate amino acid sequence;
generate, by the trained predictive protein language NLP system, a prediction including one or more biophysiochemical properties for the candidate amino acid sequence; and
display, on a display screen of a device, the predicted one or more biophysiochemical properties for the candidate amino acid sequence.

26. (canceled)

27. The computer program product of claim 25, further comprising generating concatenated representations of sequence and categorical feature embeddings from the first phase of training and providing the concatenated representations of sequence and categorical feature embeddings to the second neural network for the second phase of training.

28. (canceled)

29. The computer program product of claim 25, wherein the biophysiochemical property is binding affinity of a TCR to an epitope.

30. The computer program product of claim 25, further comprising:
training, in the first phase, the predictive protein language NLP system using a diversified protein sequence dataset that has undergone individual amino acid-level tokenization, n-mer tokenization or sub-word tokenization of respective protein sequences.

31. The computer program product of claim 30, further comprising:
training, in the first phase, the predictive protein language NLP system using the diversified protein sequence dataset, wherein about 10-20% (preferably 12-17% or 15%) of the individual amino acids in the diversified protein sequence dataset are masked.
